(19) United States
(12) Patent Application Publication
(10) Pub. No.: US 2022/0025004 A1
(43) Pub. Date: Jan. 27, 2022

(54) COMPOSITIONS AND METHODS RELATED TO SITE-SPECIFIC IDENTIFICATION OF RNA MODIFICATIONS

(71) Applicant: The University of Chicago, Chicago, IL (US)

(72) Inventors: Chuan He, Chicago, IL (US); Ralph R. Weichselbaum, Chicago, IL (US); Dali Han, Beijing (CN); Meng Xu, Beijing (CN)

(21) Appl. No.: 17/311,448

(22) PCT Filed: Dec. 20, 2019
(86) PCT No.: PCT/US2019/068001

§ 371 Date: Jun. 7, 2021

Related U.S. Application Data
(60) Provisional application No. 62/782,906, filed on Dec. 20, 2018.

(51) Int. Cl. C07K 14/47; A61P 35/00; A61K 45/06

(52) U.S. Cl. CPC C07K 14/4703; A61K 45/06; A61P 35/00

(57) ABSTRACT

The present disclosure provides compositions and methods related to cancer immunotherapy. In particular, the present disclosure identifies YTH N6-Methyladenosine RNA Binding Protein 1 (YTHDF1) as a novel therapeutic target for cancer immunotherapy. Embodiments of the present disclosure provide methods of enhancing cancer immunotherapy that involve attenuating YTHDF1 activity in various cells of the immune system (e.g., APCs) to induce a sufficient and lasting antitumor immune response.

### CROSS REFERENCE TO RELATED APPLICATION

This application claims priority to and the benefit of U.S. Provisional Patent Application No. 62/782,906 filed Dec. 20, 2018, which is incorporated herein by reference in its entirety for all purposes.

### GOVERNMENT FUNDING

This invention was made with government support under HG008935, awarded by the National Institutes of Health. The government has certain rights in the invention.

### FIELD

The present disclosure provides compositions and methods related to cancer immunotherapy. In particular, the present disclosure identifies YTH N6-Methyladenosine RNA Binding Protein 1 (YTHDF1) as a novel therapeutic target for cancer immunotherapy. Embodiments of the present disclosure provide methods of enhancing cancer immunotherapy that involve attenuating YTHDF1 activity in various cells of the immune system (e.g., APCs) to induce a sufficient and lasting antitumor immune response.

### BACKGROUND

Spontaneous T cell priming against tumor neoantigens is critical for the clinical efficacy of immunotherapies. However, in many patients, neoantigen recognition is insufficient to induce the lasting T cell response required for complete tumor rejection. Identifying molecular pathways that influence the immunoreactivity to tumor neoantigen could provide targets for improving the response to immunotherapy. For example, m⁶A, the most abundant internal mRNA modification, is responsible for posttranscriptional regulation of mRNA in diverse cell types. Additionally, m⁶A can affect mRNA translation efficiency via the m⁶A-binding protein YTHDF1. Dysregulation of m⁶A pathway components could trigger aberrant oncogene expression, revealing a link between m⁶A and tumorigenesis. As most previous reports focused on tumor-intrinsic oncogenic pathways, potential roles of the mRNA m⁶A modification in host antitumor immune responses are unknown. Further, the roles of various m⁶A reader proteins in cancer remain largely unexplored.

### SUMMARY

Embodiments of the present disclosure include a method for enhancing cancer immunotherapy treatment comprising attenuating activity of YTH N6-Methyladenosine RNA Binding Protein 1 (YTHDF1) in a subject receiving treatment with an anticancer agent. In some embodiments, YTHDF1 activity is attenuated in an antigen presenting cell (APC). In some embodiments, YTHDF1 activity is attenuated in a dendritic cell (DC). In some embodiments, attenuation of YTHDF1 activity reduces expression of lysosomal cathepsins. In some embodiments, the anticancer agent is an immune checkpoint inhibitor. In some embodiments, the immune checkpoint inhibitor is at least one of a PD-L1 antibody, a PD-1 antibody, a CTLA4 antibody, a CSG1 antibody, an IDO inhibitor, Pembrolizumab (Keytruda), Nivolumab (Opdivo), Cemiplimab (Libtayo), Atezolizumab (Tecentriq), Avelumab (Bavencio), Durvalumab (Imfinzi), and Ipilimumab (Yervoy).

In some embodiments, YTHDF1 activity is attenuated using at least one of antibodies and any derivatives thereof, antibody-drug conjugates, fusion proteins, small molecules, dsRNA, siRNA, anti-sense technology, aptamers, and gene editing technology (e.g., CRISPR-based methods).

Embodiments of the present disclosure also include a composition for treating cancer comprising an anticancer agent, and a YTH N6-Methyladenosine RNA Binding Protein 1 (YTHDF1) inhibitor. In some embodiments, the anticancer agent is an immune checkpoint inhibitor selected from a PD-L1 antibody, a PD-1 antibody, a CTLA4 antibody, a CSG1 antibody, an IDO inhibitor, Pembrolizumab (Keytruda), Nivolumab (Opdivo), Cemiplimab (Libtayo), Atezolizumab (Tecentriq), Avelumab (Bavencio), Durvalumab (Imfinzi), and Ipilimumab (Yervoy). In some embodiments, the YTHDF1 inhibitor is selected from antibodies and any derivatives thereof, antibody-drug conjugates, fusion proteins, small molecules, dsRNA, siRNA, anti-sense technology, aptamers, and gene editing technology (e.g., CRISPR-based methods).

Embodiments of the present disclosure also include methods for enhancing cancer immunotherapy treatment comprising attenuating activity of one or more lysosomal cathepsins in a subject receiving treatment with an anticancer agent. In some embodiments, lysosomal cathepsin activity is attenuated using at least one of antibodies and any derivatives thereof, antibody-drug conjugates, fusion proteins, small molecules, dsRNA, siRNA, anti-sense technology, aptamers, and gene editing technology (e.g., CRISPR-based methods). In some embodiments, lysosomal cathepsin activity is attenuated using one or more of E64, CA-074, and CASIII.

Embodiments of the present disclosure include use of a YTH N6-Methyladenosine RNA Binding Protein 1 (YTHDF1) attenuating agent for treating cancer (e.g., melanoma, breast cancer, lung cancer, ovarian cancer, brain cancer, liver cancer, cervical cancer, colon cancer, colorectal cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, and leukemia). Embodiments of the present disclosure also include use of an agent attenuating YTHDF1 activity in the preparation of a composition and/or a medicament for treating cancer.

Embodiments of the present disclosure include use of an agent attenuating YTHDF1 activity in combination with an immunotherapy for treating cancer. Embodiments also include use of an agent attenuating YTHDF1 activity in combination with an immunotherapy in the preparation of a composition and/or a medicament for treating cancer.

Embodiments of the present disclosure include use of an agent attenuating YTHDF1 activity in combination with an immune checkpoint inhibitor (e.g., a PD-L1 antibody, a PD-1 antibody, a CTLA4 antibody, a CSG1 antibody, an IDO inhibitor, Pembrolizumab (Keytruda), Nivolumab (Opdivo), Cemiplimab (Libtayo), Atezolizumab (Tecentriq), Avelumab (Bavencio), Durvalumab (Imfinzi), and Ipilimumab (Yervoy)) for treating cancer. Embodiments also include use of an agent attenuating YTHDF1 activity in combination with an immune checkpoint inhibitor in the preparation of a composition and/or a medicament for treating cancer.

In accordance with the above embodiments, YTHDF1 activity is attenuated using at least one of antibodies and any derivatives thereof, antibody-drug conjugates, fusion proteins, small molecules, dsRNA, siRNA, anti-sense technology, aptamers, and gene editing technology (e.g., CRISPR-based methods).

Embodiments of the present disclosure include use of an YTHDF1 attenuating agent for decreasing/repressing the expression (e.g., translational efficiency) of lysosomal cathepsins (cathepsin B, cathepsin L, cathepsin D).

Embodiments of the present disclosure include use of an agent attenuating lysosomal cathepsin activity in combination with immunotherapy (e.g., an immune checkpoint inhibitor) for treating cancer. Embodiments also include use of a YTHDF1 attenuating agent and/or an agent attenuating lysosomal cathepsin activity for one or more of the following: enhancing immunosurveillance; increasing CD8+ cytotoxic T cells in tumor; increasing CD8+ T cells against tumor neoantigen; reducing infiltration of myeloid-derived suppressor cells (MOSC) in tumor; increasing cross-priming ability of APCs (DC) (e.g., those induced by CD8a+ DCs and/or CD11b+ DC); enhancing the cross-presentation of tumor antigens on DCs; and enhancing the antitumor response of immune checkpoint blockade (e.g., anti-PD-L1 antibody).

### BRIEF DESCRIPTION OF THE DRAWINGS

FIGS. 1A-1E: Ythdf1^{-/-} mice show effective tumor control dependent on CD8⁺ T cells. A-B, WT or Ythdf1^{-/-} mice were injected subcutaneously (s.c.) with 10⁶ B16-OVA cells. Tumor growth (A) and survival (B) were monitored. Mice with tumor volumes less than 200 mm³ are considered to be surviving. One of three representative experiments is shown. C, WT or Ythdf1^{-/-} mice were injected s.c. with 10⁶ MC38 cells. Tumor growth was monitored. One of three representative experiments is shown. D, Percentage of tumor-infiltrating T cells and NK cells at day 12 post tumor inoculation. E, WT or Ythdf1^{-/-} mice were injected s.c. with 10⁶ B16-OVA cells. 200 μg of CD8− or NK-depleting antibody were administered twice a week starting on day 0. Tumor size was monitored over time. The number of mice for each experiment is as follow: n=10 per group (A); n=7 (WT), n=6 (Ythdf1^{-/-}) (B); n=5 per group (C); n=4 per group (D); n=6 (WT), n=4 (Ythdf1^{-/-}), n=6 (Ythdf1^{-/-}+anti-NK1.1), n=7 (Ythdf1^{-/-}+anti-CD8) (E). Data are mean±s.e.m. and were analyzed by two-tailed unpaired Student's t-test (A, C-E) or log-rank (Mantel-Cox) test (B).

FIGS. 2A-2I: Cross-priming capacity of DC is enhanced in Ythdf1^{-/-} mice. A-C, WT or Ythdf1^{-/-} mice were injected s.c. with 10⁶ B16-OVA cells. The frequency of tumor-infiltrating OVA-Specific CD8⁺ T cells was assessed 12 days post tumor inoculation (A-B). Six days post tumor inoculation, lymphocytes from DLN were isolated and stimulated with 10 μg/ml OTI peptide. IFN-γ-producing cells were enumerated by ELISPOT assay (C). D, WT or Ythdf1^{-/-} mice were injected s.c. with 10⁶ MC38 cells. Six days post tumor inoculation, lymphocytes from DLN were isolated and stimulated with irradiated MC38 cells for 48 hours. E, Flt3L-DCs were co-cultured with necrotic B16-OVA overnight, and B220⁻ CD11c⁺ cells were purified and co-cultured with OT-I T cells. IFN-γ production was assessed by IFN-γ cytometric bead array. Data are representative of three independent experiments performed in six technical replicates. F, 6 days after tumor inoculation, CD8⁺ or CD11b⁺ DCs were sorted from draining LNs. DCs were co-cultured with isolated OT-I cells for 3 days and analyzed by IFN-γ CBA. G-H, Formation of H-2K^{b}-STINFEKL on tumor-infiltrating DCs from B16-OVA tumor-bearing WT and Ythdf1^{-/-} mice (G). Mean fluoresce intensity (MFI) is shown (H). I, WT mice were transferred with WT or Ythdf1^{-/-} bone marrow cells mixed (BMCs) with Zbtb46-DTR BMCs with a 1:1 ratio. Six weeks after bone marrow chimera reconstitution, mice were injected s.c. with 1×10¹ B16-OVA cells. 400 ng DT was administrated on the same day (+DT). Tumor size was monitored over time. The number of mice for each experiment is as follow:n=5 (WT), n=4 (Ythdf^{-/-}) (B); n=4 per group (C, H, 1); n=3 per group (D, F). Data are mean±s.e.m. and were analyzed by two-tailed unpaired Student's t-test. Data are representative of two independent experiments (A, G).

FIGS. 3A-3E: Transcriptome-wide identification and analysis of the YTHDF1-binding sites. A, Volcano plots of genes with differential translational efficiency in WT and Ythdf1^{-/-} Flt3L-DCs. Transcripts with significant YTHDF1 binding sites in 3′ UTR were marked with yellow circles. P values were calculated by likelihood ratio test and adjusted by Benjamini & Hochberg method; n=2 independent biological replicates. B, Cumulative distribution log 2FoldChange of translational efficiency between WT and Ythdf1^{-/-} Flt3L-DCs. P values were calculated by two-sided Kolmogorov-Smirnov test; n=2 independent biological replicates. Box-plot elements: center line, median; box limits, upper and lower quartiles; whiskers, 1-99%. C, Metagene-plot depicting nearly unchanged m⁶A peak distribution and similar consensus motifs in WT and Ythdf1^{-/-} Flt3L-DCs. P values of consensus motifs were generated by HOMER. D, KEGG enrichment analysis of genes with significant decreased translation efficiency and YTHDF1 binding sites in 3′ UTR (n=204). One-tail hypergeometric test was used to determine statistical significance of enrichment. E. Heatmap showing the translational efficiency of lysosome genes, co-stimulatory/inhibitory molecules (signal 2) and cytokines (signal 3) in WT and Ythdf1^{-/-} Flt3L-DCs.

FIGS. 4A-4F: YTHDF1 is involved in the active translation of proteases for excessive antigen degradation. A, Representative histogram plots showing expressions of cathepsins on splenic CD8α⁺ and CD11b⁺ cDCs from WT and Ythdf1^{-/-} mice. B, WT mice were injected s.c. with 10⁶ B16-OVA cells. After 11 days, tumor-bearing mice were injected with DMSO as vehicle control (CTR) or E64 intratumorally (5 μM or 50 μM). Tumor growth was monitored over time. C. WT or Ythdf1^{-/-} mice were injected s.c. with 10⁶ B16-zsGreen-OT1 cells. The PDL1 expression on zsGreen⁺ tumor cells is shown. D, WT or Ythdf1^{-/-} mice (n=5/group) were injected s.c. with 10⁶ B16-OVA cells. 200 μg of anti-PDL1 antibody were administered on day 8 and day 15. Percentage of mice with tumor regression were monitored over time and represented as percent tumor-free survival. (E-F) Tissue sections were characterized by immunohistochemical staining for CD8 and YTHDF1. Dash line delineates the edge of tumor area. Asterisk marks the stroma tissues. Representative YTHDF1 low (Patient 1) and YTHDF1 high (Patient 5) specimens are shown (E). Scale bars, 100 μm. F, Correlations between YTHDF1 in stroma area and CD8⁺ infiltrates are shown (n=22 patients). Data are representative of two independent experiments (A, C); One of three representative images per tumor was shown (E). The number of mice for each experiment is as follow: n=4 per group (b); n=5 (WT), n=5 (Ythdf1^{-/-}), n=6 (WT+anti-PD-L1), n=7 (Ythdf1^{-/-}+anti-PD-L1) (D, F). Data are mean±s.e.m. and were analyzed by two-tailed unpaired Student's t-test (B, F) or log-rank (Mantel-Cox) test (D).

FIGS. 5A-5C: Deletion efficacy of Ythdf1^{-/-} mice. A-B, Off-target analysis of the CRISPR/Cas9 system in Ythdf1^{-/-} mice. (A) Ythdf1^{-/-} sgRNA targeting sites and four putative off-target sites were amplified. (B) PCR products of Ythdf1^{-/-} mice and WT mice were mixed and digested by T7EI. The PCR product from WT mice was used as negative control. C, Immunoblot assays were shown to validate YTH protein expression level changes in Ythdf1^{-/-} DCs. Data are representative of one experiment (A, B) and two independent biological replications for (C).

FIGS. 6A-6E: Characterizations of immune phenotypes of Ythdf1-deficient mice. a-E, WT or Ythdf1^{-/-} mice were injected s.c. with 10⁶ B16-OVA cells. (A. B) The frequency of tumor infiltrating MDSC (Ly6c⁺CD11b⁺) cells was assessed 12 days post tumor inoculation. (C, D) The percentages of Treg in spleen, draining lymph node (DLN) and tumor are shown. (E) Degranulation of tumor NK cells in response to in vitro re-stimulation with PMA/ionomycin. Data are representative of two independent experiments (A, C). The number of mice for each experiment is as follow: n=2 (Spleen), n=2 (DLN), n=4 (Tumor) (B); n=4 per group D); n=4 (Spleen), n=7 (Tumor) (E). Data are mean±s.e.m. and were analyzed by two-tailed unpaired Student's t-test.

FIGS. 7A-7F: Cross priming of tumor neoantigen is increased in Ythdf1-deficient mice. A, Rag2^{-/-} mice were transferred with T cells isolated from WT (n=4) or Ythdf1^{-/-} mice (n=4) on day 0. Rag mice (n=5) were used as controls. On the same day, mice were injected s.c. with 5×10⁵ B16-OVA cells. Tumor growth was monitored over time. B. WT or Ythdf1^{-/-} mice were injected s.c. with 10⁶ MC38-OTIp cells. 6 days after tumor inoculation, CD8⁺ or CD11b⁺ DCs were sorted from draining LNs. DCs were co-cultured with CD8⁺ T cells isolated from naive OTI mice. Capacity of cross priming was determined by the production of IFN-7. C, WT or Ythdf1^{-/-} mice were injected s.c. with 10⁶ MC38-SIY cells. 6 days after tumor inoculation, DCs were sorted from draining LNs and co-cultured with CD8⁺ T cells isolated from naive 2C mice. Capacity of cross priming was determined by the production of IFN-γ. D, WT or Mett14-deficient GMDCs were co-cultured with B16-OVA cells. The cross-priming capacity was shown. E, WT or Ythdf1^{-/-} mice were injected s.c. with 10⁶ B16-OVA cells. Data is shown as the expression of CD80 and CD86 on tumor infiltrating DCs. F. WT or Ythdf1^{-/-} mice were injected s.c. with 10⁶ B16-OVA cells. Six days after tumor inoculation, CD8+ or CDI 1b⁺ DCs were sorted from draining LNs. DCs were pulsed with 1 μg/ml exogenous OT-I peptide and co-cultured with isolated CD8⁺ T cells from naive OTI mice for 3 days and analyzed by IFN-γ CBA. Data are representative of two independent experiments E). The number of mice for each experiment is as follow: n=3 per group (B, D); n=4 (WT), n=6 (Ythdf1^{-/-}) (C); n=4 per group (F). Data are mean±s.e.m. and were analyzed by two-tailed unpaired Student's t-test (A-C. F) or one-tailed unpaired Student's t-test (D).

FIGS. 8A-8D: The development of DCs and T cells were similar in Ythdf1^{+/+} and Ythdf1^{-/-} mice. A-B, Percentages of CD11b⁺ and CD8α⁺ DCs in lymph node (LN) and spleen are shown. C-D, Percentages of CD4⁺ and CD8⁺ T cells in lymph node (LN) and spleen are shown. No significant difference was detected between WT and Ythdf1^{-/-} mice. The number of mice for each experiment is as follow: n=4 per group (A, C, D); n=4 (WT), n=5 (Ythdf1^{-/-}) (B). Data are mean±s.e.m. and were analyzed by two-tailed unpaired Student's t-test.

FIGS. 9A-9D: In vitro functional analysis of GMCSF-induced DCs (GMDCs) generated from Ythdf1^{-/-} mice. A, The production of IL-6, CCL2 and TNFα upon stimulation of Ythdf1^{-/-} GMDCs with LPS. B-C. WT (n=4) or Ythdf1^{-/-} (n=4) mice were injected s.c. with 10⁶ B16-OTI-zsGreen cells. Percentage of tumor infiltrating zsGreen⁺ DC, six days after tumor inoculation, is shown. Data are representative of two independent experiments (B). D, Splenic DCs from WT and Ythdf1^{-/-} mice were stimulated with LPS overnight. Cross-presentation capacity of DCs in response to soluble OVA was assessed. n=3 independent experiments for (A); n=6 independent experiments for (D). Data are mean±s.e.m. and were analyzed by two-tailed unpaired Student's t-test.

FIGS. 10A-10D: Transcriptome-wide analysis of the YTHDF1 binding sites in Flt3L-DCs. A, High reproducibility of YTHDF1 RIP-seq data. For each potential YTHDF1 binding peak, the fold-enrichment of RIP/Input signal was determined for both Replicate 1 and Replicate 2. The peaks identified in both replicates were considered as high-confidence peak and indicated in red. B, Overlap of YTHDF1-binding transcripts revealed from RIP-seq of two biological replicates. C. Meta-gene analysis to show the distribution of YTHDF1-binding sites along a normalized transcript. S. Distribution of YTHDF1-binding sites in transcripts.

FIGS. 11A-11G: YTHDF1-deficient GMDCs exhibit lower translational rates. A, High reproducibility of YTHDF1 RIP-seq data in GMDCs. For each potential YTHDF1 binding peak, the fold-enrichment of RIP/Input signal was determined for both Replicate 1 and Replicate 2. The peaks identified in both replicates were considered as high-confidence peak and indicated in red. B. Volcano plots of genes with differential translational efficiency in WT and Ythdf1^{-/-} GMDCs. YTHDF1 targets were marked with yellow circles. P values were calculated by likelihood ratio test and adjusted by Benjamini & Hochberg method; n=2 independent biological replicates. C, Cumulative distribution log 2FoldChange of translational efficiency between WT and Ythdf1^{-/-} GMDCs. P values were calculated by two-sided Kolmogorov-Smirnov test; n=2 independent biological replicates. D, Pie charts presenting the distribution of YTHDF1-binding sites in transcripts. E, Metagene-plot depicting nearly unchanged m⁶A peaks distribution and similar consensus motifs in WT and Ythdf1^{-/-} GMDCs. P values of consensus motifs were generated by HOMER. F. KEGG and GO enrichment analysis of YTHDF1 target genes revealed enrichment of biological functions related to innate immune system, lysosome and phagosome (n=79). One-tail hypergeometric test was used to determine statistical significance of enrichment. G, Heatmap showing translational efficiency of cathepsin genes in GMDCs and Flt3L-DCs.

FIGS. 12A-12F: Antigen degradation is reduced in Ythdf1^{-/-} mice and inhibition of protease cathepsins enhanced the cross-priming of WT DCs. A, WT and Ythdf1^{-/-} DCs were treated with Actinomycin D, RNAs collected at different time points after treatment, and mRNA levels were measured using RT-qPCR and represented as mRNA remaining after transcription inhibition (TI). NS, not significant. B, GMDCs were co-cultured with necrotic B16-OVA cells overnight. Immunoblot analysis of proteases Cathespins B/D/L (CTSB, CTSD and CTSL) in GMDCs. C. GMDCs were co-cultured with necrotic B16-OVA cells overnight and OVA degradation in BMDCs was measure by Immunoblot. D, Ex vivo purified wild-type cDCs were pre-treated with 0.04 μM cathepsin inhibitor E64 and pulsed with OVA protein for 4 h. The cross-priming capacity of DCs was compared by co-culturing DCs with cell trace violet (CTV) labeled OTI-T cells. The proliferation was measured by the dilution of CTV. E, GMDCs were pre-treated with 0.2-2 μM cathepsin inhibitor E64 and co-cultured with B16-OVA cells. The cross-priming capacity of DC was compared by co-culturing DCs with isolated CD8⁺ T cells from naive OTI mice and analyzed by IFN-γ cytometric bead array. F. Flt3L-DCs were pre-treated with cathepsin inhibitor CA-074 or/and cathepsin L inhibitor III (CASIII), followed by co-culturing with necrotic B16-OVA cells. Synergistic inhibition effects were observed. The cross-priming capacity of DC was determined; n=2-3 independent experiments (A. D): n=3-4 independent experiments (E. F).

FIG. 13: IFNγ within tumor tissues is responsible for the upregulation of PD-L1 in Ythdf1^{-/-} mice. Tumor-bearing mice were treated with 50 μg anti-IFNγ mAb intratumorally (i.t.) and PD-L1 expression on tumor cells is shown. The number of mice was n=3 (WT), n=5 (Ythdf1^{-/-}), and n=3 (Ythdf1^{-/-}+anti-IFNγ).

### DETAILED DESCRIPTION

The present disclosure provides compositions and methods related to cancer immunotherapy. In particular, the present disclosure identifies YTH N6-Methyladenosine RNA Binding Protein 1 (YTHDF1) as a novel therapeutic target for cancer immunotherapy. Embodiments of the present disclosure provide methods of enhancing cancer immunotherapy that involve attenuating YTHDF1 activity in various cells of the immune system (e.g., APCs) to induce a sufficient and lasting antitumor immune response.

Emerging evidence suggests important roles of tumor neoantigens in generating spontaneous antitumor immune responses and predicting clinical responses to immunotherapies. Despite the presence of numerous neoantigens, complete tumor elimination rarely occurs in many patients, often times due to failures in mounting a sufficient and lasting antitumor immune response. Embodiments of the present disclosure demonstrate that durable neoantigen-specific immunity is regulated by messenger RNA (mRNA) N⁶-methyladenosine (m⁶A) methylation through the m⁶A-binding protein YTHDF1. For example, in contrast to wild-type mice, Ythdf1-deficient (Ythdf1^{-/-}) mice exhibit an elevated antigen-specific CD8⁺ T cell antitumor response. Loss of YTHDF1 in classical dendritic cells (cDCs) enhanced the cross-presentation of tumor antigen and the cross-priming of CD8⁺ T cells in vivo. Mechanistically, transcripts encoding lysosomal proteases are marked by m⁶A and recognized by YTHDF1. Binding of YTHDF1 to these transcripts elevates translation of lysosomal cathepsins in DCs, with the inhibition of cathepsins markedly enhancing cross-presentation of the wild-type DCs. Furthermore, the therapeutic efficacy of PD-L1 checkpoint blockade is enhanced in Ythdf1^{-/-} mice, implicating YTHDF1 as a new potential therapeutic target in cancer immunotherapy.

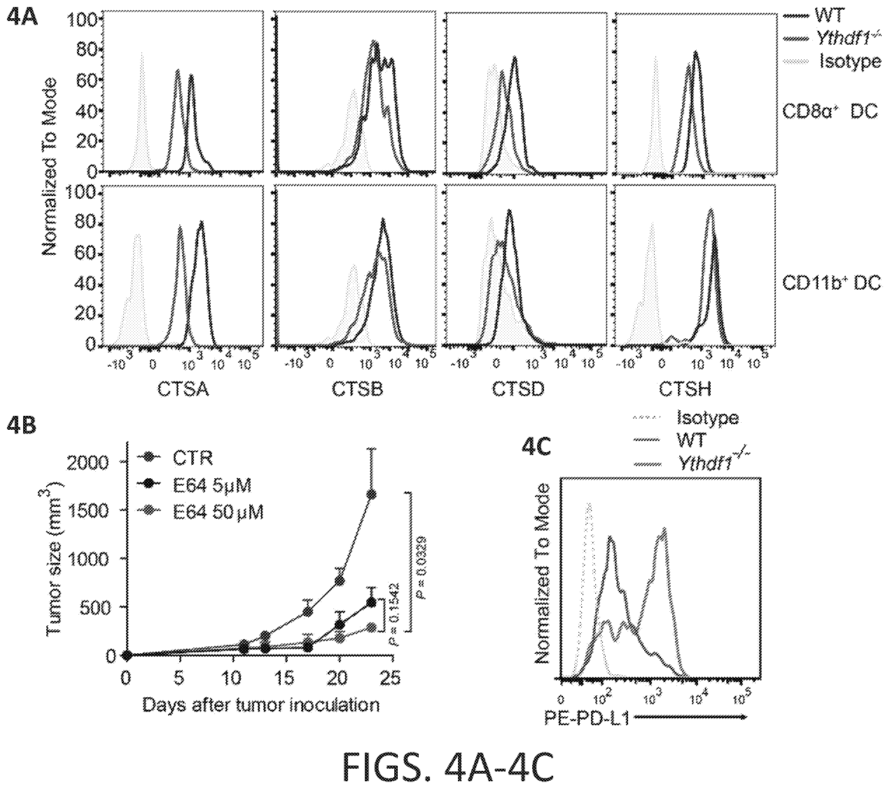

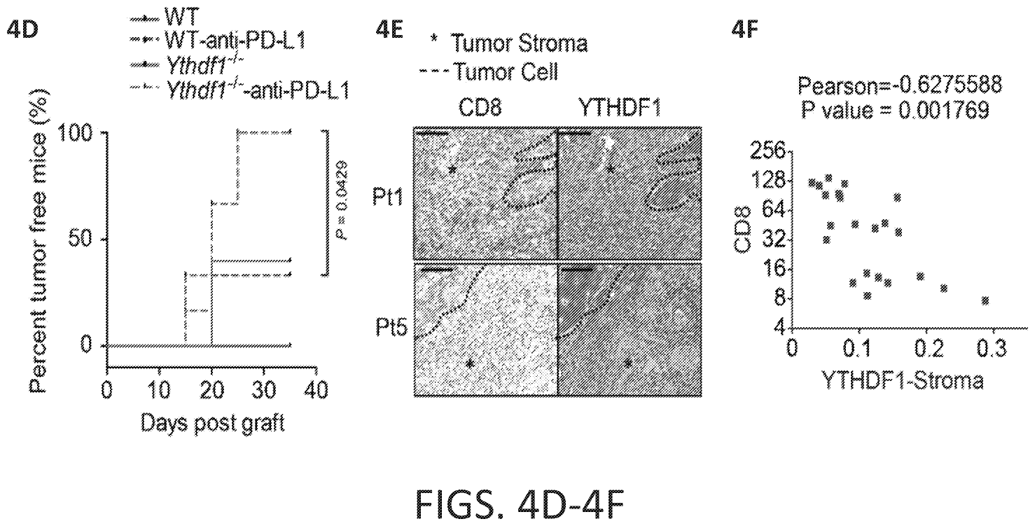

Among other findings, embodiments of the present disclosure discovered that colon cancer patients with low expression of YTHDF1 in tumor stroma tended to have higher number of CD8⁺ cells, while biopsies with notably high expression of YTHDF1 in the tumor stroma lacked CD8⁺ cell infiltrates (see, e.g., FIGS. 4E-4F), demonstrating the correlation between reduced YTHDF1 expression and T-cell activation in the inflamed tumor microenvironment.

Additionally, as would be recognized by one of ordinary skill in the art based on the present disclosure, clinical responses to immunotherapy do not always correlate with the neoantigen burden. Tumors can evade immune recognition despite expressing neoantigens. Results of the present disclosure reveal that the m⁶A-marked mRNAs encoding lysosomal proteases are recognized by YTHDF1 in DCs. YTHDF1 binding promotes translation of lysosomal proteases, suppressing the cross-presentation of engulfed tumor neoantigens, which represents a previously unrecognized mechanism of immune evasion. Results of the present disclosure demonstrate that YTHDF1 is a therapeutic target for immunotherapy, and can also be modulated in combination with emerging checkpoint inhibitors or DC vaccines.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict. the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can.” “contain(s),” and variants thereof, as used herein, are intended to be open-ended transitional phrases, terms. or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

“Antibody” and “antibodies” refer to monoclonal antibodies, multispecific antibodies, bifunctional antibodies, human antibodies, humanized antibodies (fully or partially humanized), animal antibodies (such as, but not limited to, antibodies obtained or derived from a bird (for example, a duck or a goose), a shark, a whale, and a mammal, including a non-primate (for example, a cow, a pig, a camel, a llama, a horse, a goat, a rabbit, a sheep, a hamster. a guinea pig, a cat, a dog, a rat, a mouse, etc.) or a non-human primate (for example, a monkey, a chimpanzee, etc.), recombinant antibodies, chimeric antibodies, single-chain fragment variable (“scFv”), single chain antibodies, single domain antibodies. Fab fragments, F(ab′) fragments, F(ab′)₂ fragments, disulfide-linked fragment variable (“sdFv”), and anti-idiotypic (“anti-Id”) antibodies, dual-domain antibodies, dual variable domain (DVD) or triple variable domain (TVD) antibodies (see, e.g., Wu et al., Nature Biotechnology, 25(11): 1290-1297 (2007), and International Patent Application Publication No. WO 2001/058956)), and functionally active epitope-binding fragments of any of the above. The term “bifunctional antibody,” as used herein, refers to an antibody that comprises a first arm having a specificity for one antigenic site and a second arm having a specificity for a different antigenic site, i.e., the bifunctional antibodies have a dual specificity.

The terms “antibody fragment” and “antibody fragments” refer to a portion of an intact antibody comprising the antigen-binding site or variable region. The portion does not include the constant heavy chain domains (i.e. CH2. CH3 or CH4, depending on the antibody isotype) of the Fe region of the intact antibody. Examples of antibody fragments include, but are not limited to, Fab fragments, Fab′ fragments, Fab′-SH fragments. F(ab′)₂ fragments. Fd fragments, Fv fragments, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

As used herein, “diagnosis” and similar terms refer to the identification of a particular disease.

“Label” and “detectable label” generally refers to a moiety attached, directly or indirectly, to an analyte-binding molecule (e.g., antibody or analyte-reactive fragment thereof) or an analyte to render the reaction between the analyte-binding molecule (e.g., antibody or analyte-reactive fragment thereof, a nucleic acid probe, etc.) and the analyte detectable, and the an analyte-binding molecule (e.g., antibody or analyte-reactive fragment thereof) or analyte so labeled is referred to as “detectably-labeled.” A label can produce a signal that is detectable, such as by visual or instrumental means. In some aspects, a label can be any signal-generating moiety, and sometimes is referred to herein as a reporter group. As used herein, the label (or signal-generating moiety) produces a measurable signal which is detectable by external means, such as by the measurement of electromagnetic radiation, and, depending on the system employed, the level of signal can vary to the extent the label is in the environment of the solid support (e.g., an electrode, microparticle or bead).

“Sample,” “biological sample,” “test sample,” “specimen.” “sample from a subject,” and “patient sample” as used herein may be used interchangeable and may be a sample of blood, tissue, urine, serum, plasma, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration. centrifugation, inactivation of interfering components, addition of reagents, and the like, to modify the character of the sample in some manner as discussed herein or otherwise as is known in the art.

Any cell type, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, sputum, stool, tears, mucus, saliva, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include lymph fluid, mammary tissue, epithelial tissue, ascetic fluid, gynecological fluid, urine, peritoneal fluid, cerebrospinal fluid, a fluid collected by vaginal rinsing, or a fluid collected by vaginal flushing, breast tissue, ovarian tissue, brain tissue, bone tissue, genital tract tissue, gastrointestinal tract tissue, nervous system tissue, lung tissue, prostate tissue, and immune system tissue. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary.

As used herein, the terms “subject” and “patient” are used interchangeably irrespective of whether the subject has or is currently undergoing any form of treatment. As used herein, the terms “subject” and “subjects” refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc.) and a human). In some aspects, the subject is a human.

The terms “treat,” “treated,” or “treating,” as used herein, refer to a therapeutic method wherein the object is to slow down (lessen) an undesired physiological condition. disorder or disease, or to obtain beneficial or desired clinical results. In some aspects of the present disclosure, beneficial or desired clinical results include, but are not limited to, alleviation of symptoms; diminishment of the extent of the condition, disorder or disease: stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment also includes prolonging survival as compared to expected survival if not receiving treatment.

Unless otherwise defined herein, scientific and technical terms used in connection with the present disclosure shall have the meanings that am commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Compositions and Methods

Embodiments of the present disclosure include methods for treating cancer in a subject by attenuating activity of YTH N6-Methyladenosine RNA Binding Protein 1 (YTHDF1). In accordance with these embodiments, the method can include attenuating YTHDF1 activity in an immune cell, including but not limited to, an antigen presenting cell (APC) such as a dendritic cell (DC). Attenuating YTHDF1 activity in a DC can be used to treat any type of cancer, including but not limited to, melanoma, breast cancer, lung cancer, ovarian cancer, brain cancer, liver cancer, cervical cancer, colon cancer, colorectal cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, and leukemia.

In some embodiments, an inhibitor of YTHDF1 activity can be any molecule or agent that functions to reduce, attenuate, or eliminate YTHDF1 activity, including but not limited to, antibodies and any derivatives thereof, antibody-drug conjugates, fusion proteins, small molecules, dsRNA, siRNA, anti-sense technology, aptamers, and gene editing technology (e.g., CRISPR-based methods). As would be recognized by one of ordinary skill in the art based on the present disclosure, inhibiting or attenuating YTHDF1 activity can include genetic means (e.g., gene silencing or knockdown using gene editing tools such as CRISPR-Cas9, TALENs, ZFNs, and the like), as well as antisense nucleotides that target mRNA stability or expression (e.g., dsRNA, siRNA, anti-sense aptamers, and the like). Inhibiting or attenuating YTHDF1 activity can also include methods that interfere with the activity of YTHDF1 protein itself, such as antibodies that bind to YTHDF1 and inhibit one or more aspects of its function. as well as pharmacological or small molecule inhibition that interferes with the stability or activity of YTHDF1.

In some embodiments, the attenuation of YTHDF1 activity reduces expression and/or activity of lysosomal cathepsins and treats the cancer. Cathepsins are lysosomal proteases that belong to the papain family. More than a dozen cathepsins have been identified in various organisms (e.g., cathepsin A, cathepsin B, cathepsin C, cathepsin D, cathepsin E, cathepsin F, cathepsin G, cathepsin H, cathepsin J, cathepsin K, cathepsin L, cathepsin O, cathepsin S, cathepsin T, cathepsin V, cathepsin W, cathepsin Y, and cathepsin Z). Previous reports have demonstrated that increased lysosomal enzymatic activity and secretion of the hydrolases into the extracellular space promotes tumor growth and migration, angiogenesis, and metastasis, and several members of the cathepsin family of proteases have been implicated in tumor progression.

In particular, protein expression levels of cathepsins B, D, and L have been shown to be increased in tumor. In some cases, there is a redistribution of lysosomes from perinuclear to peripheral locations in cancer cells and the lysosomal contents have been localized to the extracellular space. Additionally, secreted cathepsins, working in collaboration with matrix metalloproteases and the plasminogen activator system, can degrade the extracellular matrix (ECM), thereby promoting cellular motility, invasion, and angiogenesis. In some cases. reduction of one or more of cathepsins B, D, and L can be advantageous due to its involvement in various pathologies and oncogenic processes. In normal physiological conditions, cathepsins B. D, and L are tightly regulated in a well-coordinated manner at multiple levels. However, during malignant transformation, the regulation of cathepsins B, D, and L can be altered at multiple levels and thereby resulting in the overproduction.

In accordance with these embodiments, the method can include enhancing cancer immunotherapy treatment by attenuating activity of one or more lysosomal cathepsins (e.g., cathepsins B, C, L, F, H, K, O, S, V, X and W) in a subject receiving treatment with an anticancer agent. In some embodiments, lysosomal cathepsin activity can be attenuated using at least one of antibodies and any derivatives thereof, antibody-drug conjugates, fusion proteins, small molecules, dsRNA, siRNA, anti-sense technology, aptamers, and gene editing technology (e.g., CRISPR-based methods). In some embodiments, lysosomal cathepsin activity can be attenuated using one or more of E64, CA-074, and CASIII.

As would be recognized by one of ordinary skill based on the present disclosure, a cathepsin inhibitor can be any agent which inhibits the transcription of a cathepsin gene, the processing or translation of a cathepsin mRNA, or the processing, trafficking or activity of a cathepsin protein, when administered in vivo or in vitro to a mammalian cell which is otherwise competent to express active cathepsin. For example, cathepsin inhibitors include repressors, which inhibit induction and/or transcription of a cathepsin gene, and an antisense nucleotide or nucleic acid aptamer that selectively binds to a cathepsin DNA or mRNA sequence and which inhibits the transcription or translation of the cathepsin sequences. Embodiments also include competitive and non-competitive inhibitors of the activity of a cathepsin protein, such as small molecules which structurally mimic the natural substrates of a cathepsin, but which are resistant to the proteolytic activity of the enzyme.

Embodiments of the present disclosure also include treating cancer in a subject by administering an inhibitor of YTHDF1 activity. In some embodiments, the inhibitor of YTHDF1 activity is administered with an anticancer agent. In some embodiments, the anticancer agent is an immune checkpoint inhibitor. Generally, immune checkpoints are a normal part of the immune system; they prevent an immune response from being so activated that it targets healthy cells in addition to cancerous cells. Immune checkpoints occur when proteins on the surface of immune cells (e.g., T cells) recognize and bind to complementary proteins on other cells (e.g., tumor cells), which are called immune checkpoint proteins. When the checkpoint and complementary proteins bind together, they functionally deactivate the T cells, which prevents the immune system from targeting the tumor cell for destruction. Immunotherapy drugs called immune checkpoint inhibitors function by blocking checkpoint proteins from binding with their complementary proteins. This allows the T cells to target the tumor cells. Thus, embodiments of the present disclosure include compositions and methods with immune checkpoint inhibitors. Immune checkpoint inhibitors can include, but are not limited to, a PD-L1 antibody, a PD-1 antibody, a CTLA4 antibody, a CSG1 antibody, an IDO inhibitor, Pembrolizumab (Keytruda). Nivolumab (Opdivo), Cemiplimab (Libtayo), Atezolizumab (Tecentriq), Avelumab (Bavencio). Durvalumab (Imfinzi), and Ipilimumab (Yervoy).

In some embodiments, the methods include treating cancer in a subject by administering an inhibitor of YTHDF1 activity, an anticancer such as an immune checkpoint inhibitor, and further a separate anticancer agent (e.g., chemotherapeutic drugs). As would be recognized by one of ordinary skill in the art based on the present disclosure, chemotherapeutic agents, also referred to as antineoplastic agents, are used to directly or indirectly inhibit the proliferation of rapidly growing cells, typically in the context of malignancy. They are classified according to their mechanism of action and include alkylating agents, anthracyclines, cytoskeletal disruptors (e.g., taxanes), epothilones, histone deacetylase inhibitors, inhibitors of topoisomerase I, inhibitors of topoisomerase II, kinase inhibitors, nucleotide analogs and precursor analogs, peptide antibiotics, platinum-based agents, and retinoids.

Embodiments of the present disclosure include use of a YTH N6-Methyladenosine RNA Binding Protein 1 (YTHDF1) attenuating agent for treating cancer (e.g., melanoma. breast cancer, lung cancer, ovarian cancer, brain cancer, liver cancer, cervical cancer, colon cancer, colorectal cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, and leukemia). Embodiments of the present disclosure also include use of an agent attenuating YTHDF1 activity in the preparation of a composition and/or a medicament for treating cancer.

Embodiments of the present disclosure include use of an agent attenuating YTHDF1 activity in combination with an immunotherapy for treating cancer. Embodiments also include use of an agent attenuating YTHDF1 activity in combination with an immunotherapy in the preparation of a composition and/or a medicament for treating cancer.

Embodiments of the present disclosure include use of an agent attenuating YTHDF1 activity in combination with an immune checkpoint inhibitor (e.g., a PD-L1 antibody, a PD-1 antibody, a CTLA4 antibody, a CSG1 antibody, an IDO inhibitor, Pembrolizumab (Keytruda), Nivolumab (Opdivo), Cemiplimab (Libtayo), Atezolizumab (Tecentriq), Avelumab (Bavencio), Durvalumab (Imfinzi), and Ipilimumab (Yervoy)) for treating cancer. Embodiments also include use of an agent attenuating YTHDF1 activity in combination with an immune checkpoint inhibitor in the preparation of a composition and/or a medicament for treating cancer.

In accordance with the above embodiments. YTHDF1 activity is attenuated using at least one of antibodies and any derivatives thereof, antibody-drug conjugates, fusion proteins. small molecules, dsRNA, siRNA, anti-sense technology, aptamers, and gene editing technology (e.g., CRISPR-based methods).

Embodiments of the present disclosure include use of an YTHDF1 attenuating agent for decreasing/repressing the expression (e.g., translational efficiency) of lysosomal cathepsins (cathepsin B, cathepsin L, cathepsin D). Embodiments of the present disclosure include use of an agent attenuating lysosomal cathepsin activity in combination with immunotherapy (e.g., an immune checkpoint inhibitor) for treating cancer. Embodiments also include use of a YTHDF1 attenuating agent and/or an agent attenuating lysosomal cathepsin activity for one or more of the following: enhancing immunosurveillance; increasing CD8+ cytotoxic T cells in tumor; increasing CD8+ T cells against tumor neoantigen; reducing infiltration of myeloid-derived suppressor cells (MOSC) in tumor; increasing cross-priming ability of APCs (DC) (e.g., those induced by CD8a+ DCs and/or CD11b+ DC); enhancing the cross-presentation of tumor antigens on DCs; and enhancing the antitumor response of immune checkpoint blockade (e.g., anti-PD-L1 antibody).

### 3. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

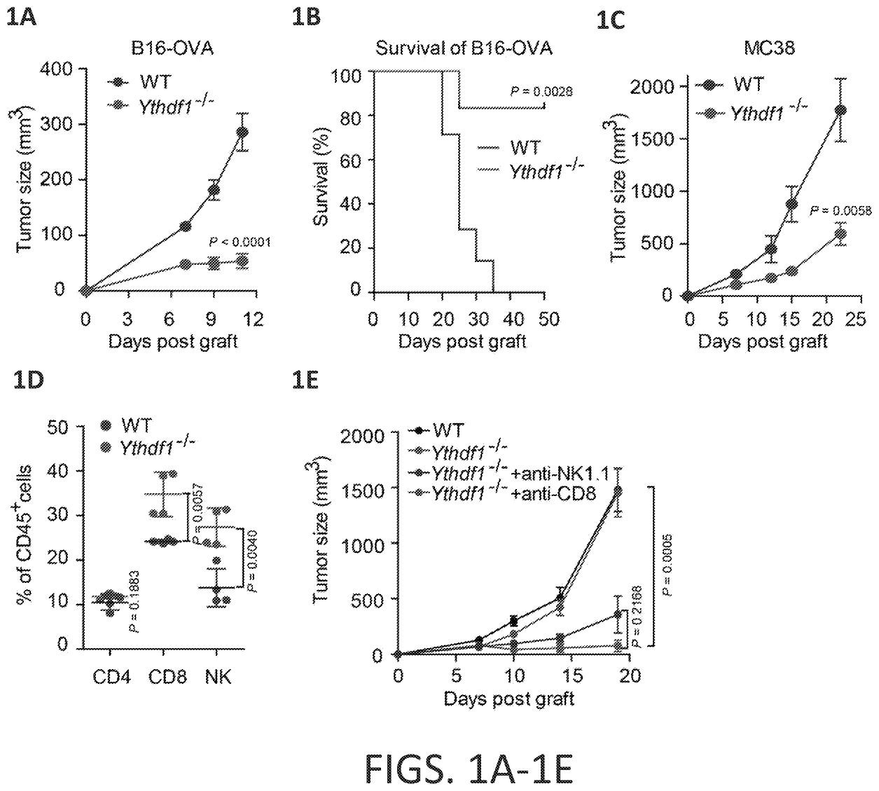

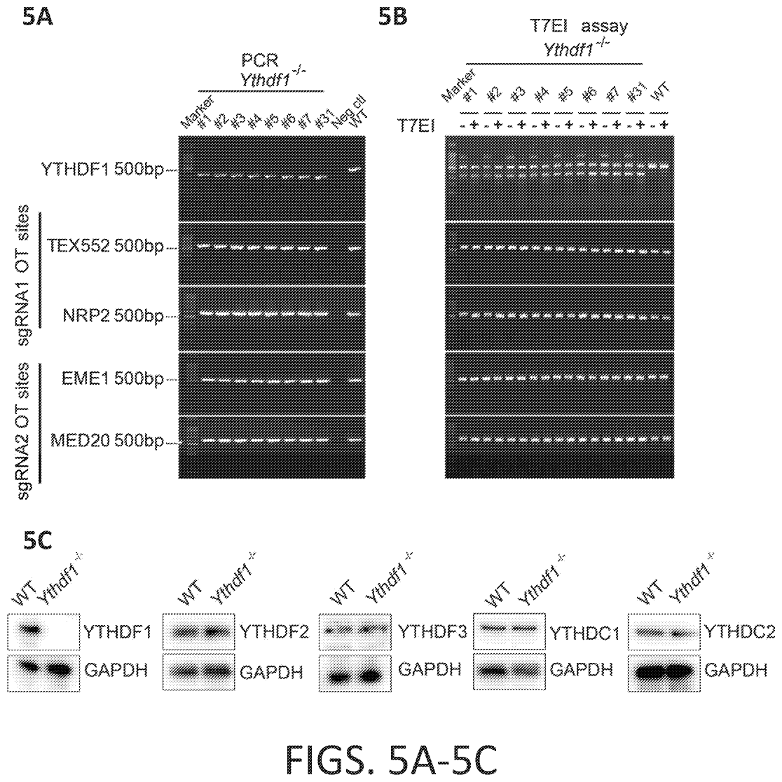

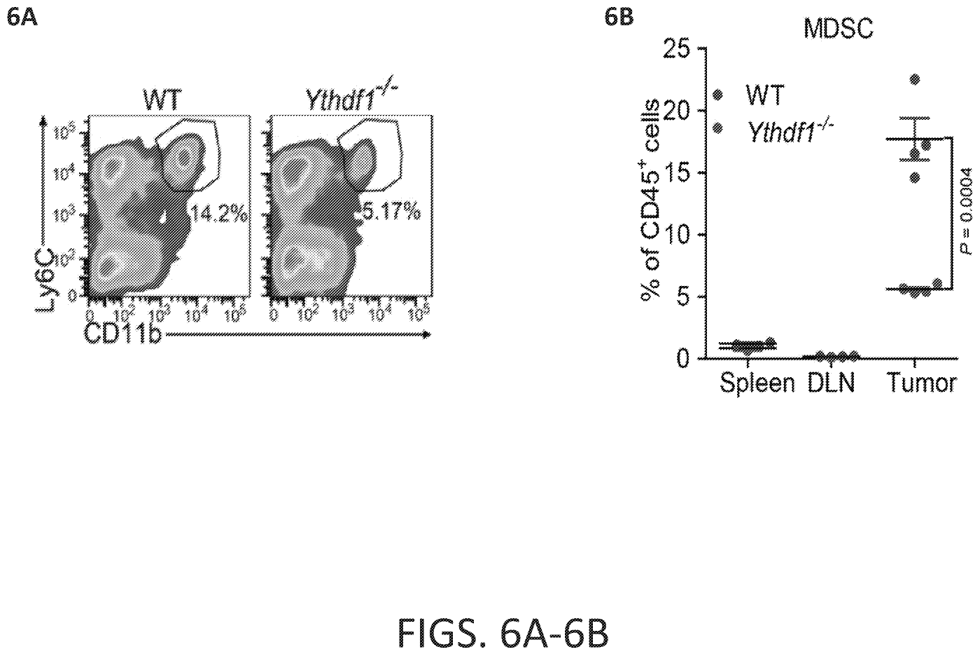

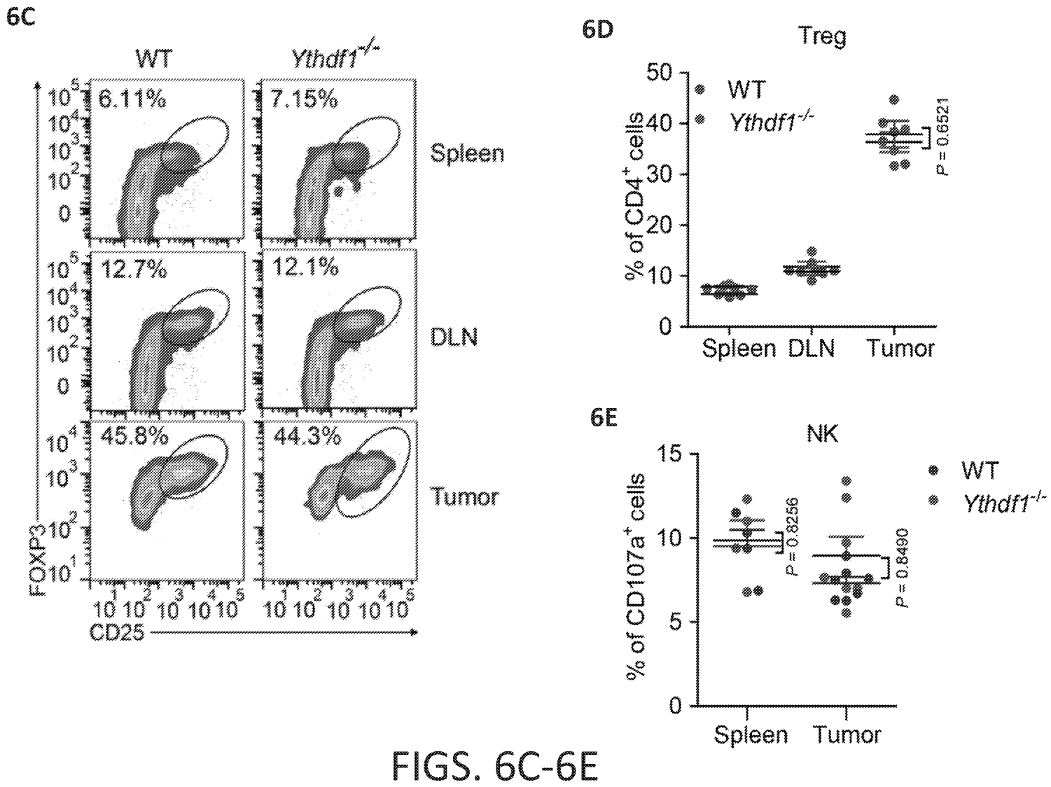

The m⁶A reader Ythdf1 knockout mice (FIGS. 5A-5C) were inoculated with ovalbumin (OVA)-expressing B16 melanoma cells subcutaneously (s.c.) along with WT control mice. Compared to WT mice. Ythdf1^{-/-} mice showed slower growth of B16-OVA tumors and prolonged survival (FIGS. 1A-1B). These findings were tested in an MC38 colon carcinoma model, which has been reported to have a broader neoantigen pool. A similar level of tumor inhibition in Ythdf1^{-/-} relative to WT mice was observed (FIG. 1C). Immune infiltrates were analyzed, and higher levels of CD8⁺ cytotoxic T cells and natural killer (NK) cells in tumors from Ythdf1^{-/-} mice were found compared to WT mice, suggesting that an enhanced immunosurveillance occurs in the absence of YTHDF1 (FIG. 1D). A reduced infiltration of myeloid-derived suppressor cells (MDSC) was observed in tumors of Ythdf1^{-/-} mice (FIGS. 6A-6B). whereas there was no significant difference in Treg (FIGS. 6C and 6D). Both CD8⁺ T cells and NK cells are critical for controlling tumor growth; therefore, their contributions to the anti-tumor response in Ythdf1^{-/-} mice was investigated. NK cells from WT and Ythdf1^{-/-} mice showed similar degranulation responses (FIG. 6E), and antibody-mediated depletion of NK cells had no effect on tumor growth in Ythdf1^{-/-} mice (FIG. 1E). In contrast, the anti-tumor response in Ythdf1^{-/-} mice was completely abrogated in the absence of CD8⁺ T cells (FIG. 1E). indicating that CD8⁺ T cells are important for tumor control in the Ythdf1-deficient host.

### Example 2

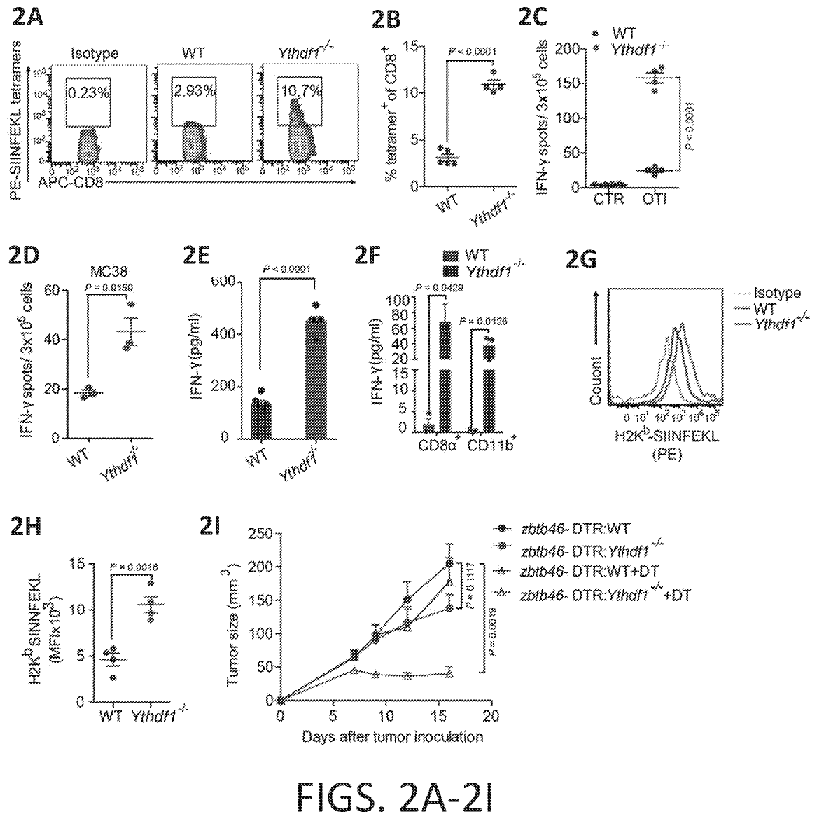

To determine whether neoantigen-specific CD8⁺ T cell responses are generated in B16-OVA tumors, the frequency of tumor-infiltrating SIINFEKL MHC-I tetramer⁺ CD8⁺ T cells in WT and Ythdf1⁺ mice was analyzed. While WT mice failed to accumulate antigen-specific CD8⁺ T cells within the tumor, Ythdf1^{-/-} showed a substantially increased CD8⁺ T cells against tumor neoantigen in vivo relative to WT mice (FIGS. 2A-2B). To investigate whether the infiltration of neoantigen-specific CD8⁺ T cells in Ythdf1^{-/-} mice is due to enhanced spontaneous CD8⁺ T cell priming at an early stage, lymphocytes from tumor-draining lymph node were stimulated in vitro, with or without the OVA-derived SIINFEKL peptide or tumor MC38 cells, and measured the tumor-specific CD8⁺ T-cell responses using IFN-γ ELISPOT. Significantly more IFN-γ spot-forming cells were present in Ythdf1^{-/-} mice than in WT mice in both B16-OVA and MC38 tumor models (FIGS. 2C-2D), indicating that YTHDF1 depletion in host cells potentiates the early steps of T-cell priming against tumor neoantigens.

### Example 3

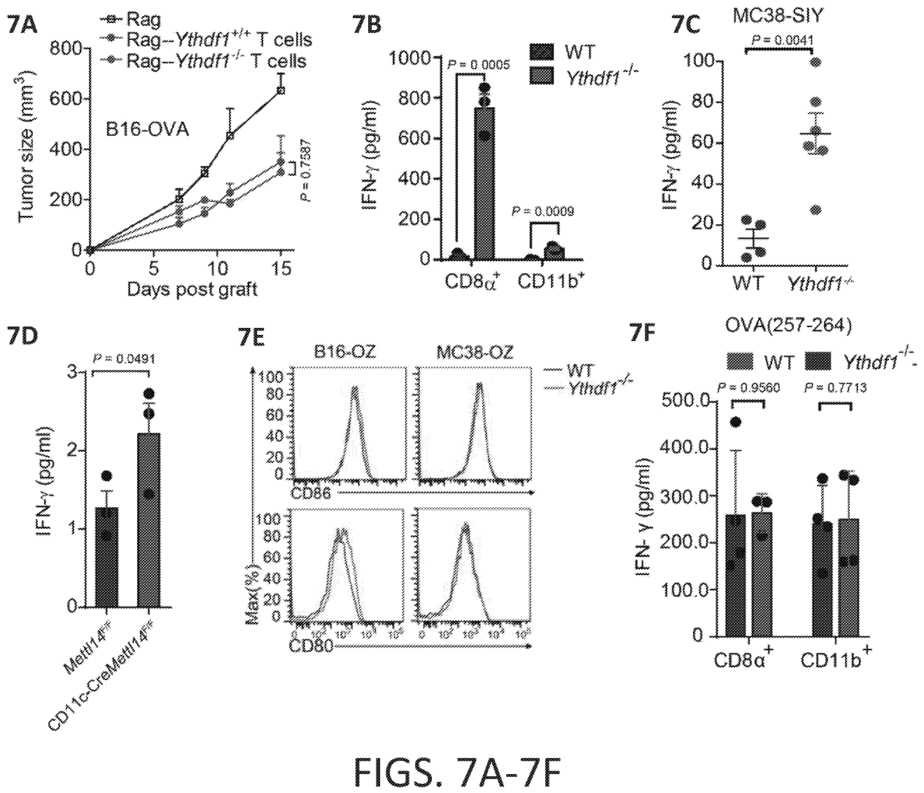

Next, loss of Ythdf1 in T cells were shown to be involved in the observed antitumor immunity (FIG. 7A). Since dendritic cells (DCs) are the main antigen-presenting cells (APCs) that cross-prime CD8⁺ T cells, experiments were conducted to test whether increased T cell priming in Ythdf1^{-/-} mice could be attributed to an improved recognition of tumor cells through an increased cross-priming ability of DCs. Flt3L-supplemented cultures were used for classical DCs (Flt3L-DC) to model how cross-presentation occurs. Flt3L-DCs were pulsed with necrotic B16-OVA in vitro and their ability to cross-prime OTI-specific TCR-transgenic OT-I T cells was evaluated. It was found that Ythdf1^{-/-} Flt3L-DCs were able to cross-prime OT-I T cells to a greater extent than WT DCs (FIG. 2E). To determine the cross-priming capacity of classical DCs (cDC) in vivo, CD8α⁺ DCs and CD11b⁺ DCs were collected from draining lymph nodes (DLNs) of B16-OVA- or MC38-OTIp-tumor bearing mice and co-cultured them with OT-I T cells. While a weak cross-priming of CD8⁺ T cells was detected in WT mice, a significantly augmented T cell cross-priming induced was observed by both CD8α⁺ DCs and CD11b⁺ DCs in Ythdf1^{-/-} mice (FIG. 2F; FIG. 7B). In addition, a less sensitive model antigen SIY was utilized and it was confirmed that the enhanced cross-presentation capacity was consistently detected in Ythdf1^{-/-} DCs (FIG. 7C). To test whether cross-priming in DCs depends on RNA m⁶A methylation in general, the cross-presentation capacity of Mettl14-deficient DCs (employing CD11c-CreMettl14^{f/f} conditional knockout mice) and WT DCs were compared. Indeed, DCs deficient in Mettl14 showed enhanced capacity in cross-presentation (FIG. 7D), reinforcing the critical role of the m⁶A-YTHDF1 axis in restricting the cross-priming capacity of DCs.

### Example 4

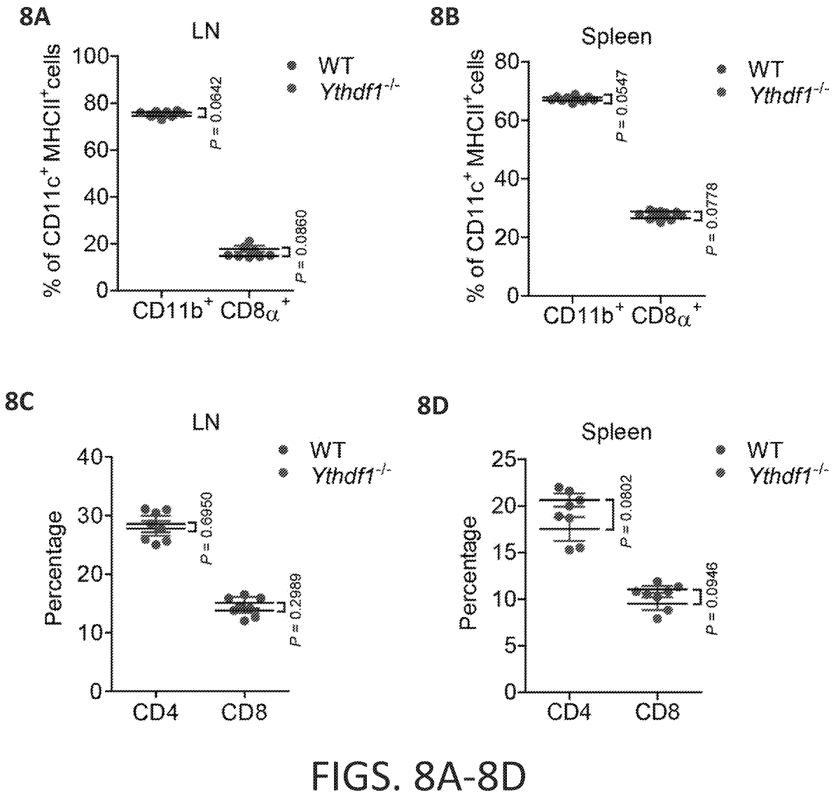

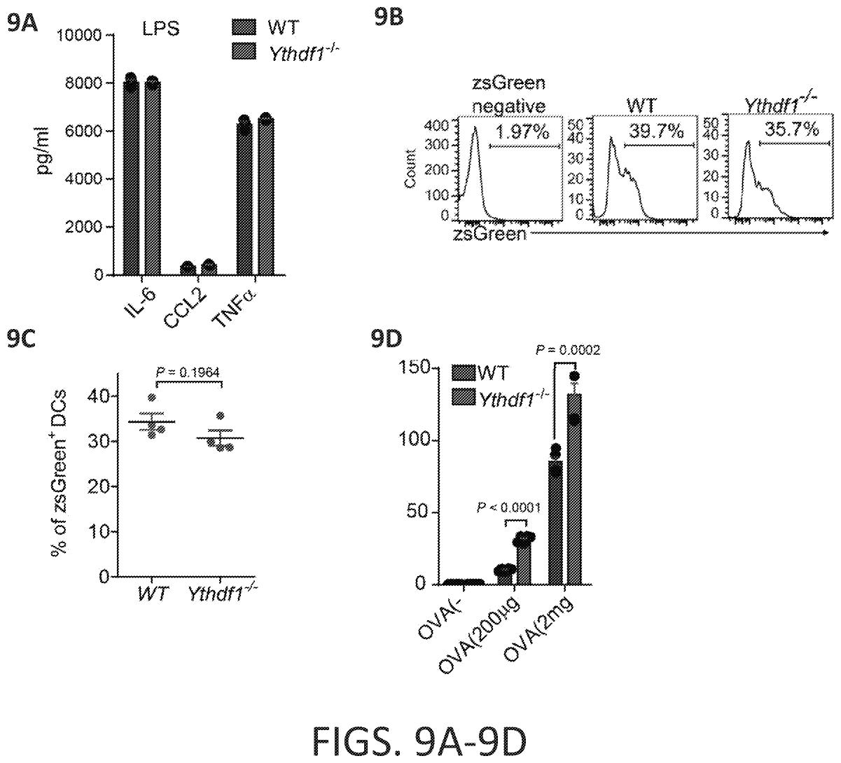

Considering the possibility that the increased cross-capacity of YTHDF1-deficient DCs for priming could be attributed to differential expression of co-stimulatory molecules, the expressions of CD80 and CD86 on DCs was evaluated. WT and Ythdf1^{-/-} DCs expressed comparable levels of CD80 and CD86, and also exhibited a similar ability to directly prime OT-I T cells with peptide stimulation (FIGS. 7E-7F). In addition, loss of YTHDF1 did not affect the composition of DC subpopulations in naive mice (FIG. 8), nor did it affect LPS-mediated DC activation (FIG. 9A). These findings suggest that loss of YTHDF1 increases cross-priming capacity of DCs, rather than impacting the development or activation of DCs.

### Example 5

To determine if YTHDF1 deficiency enhances the cross-presentation of tumor antigens on DCs, leading to better cross-priming of CD8⁺ T cells, the abundance of H-2K^{b}-SIINFEKL complexes on DCs from WT and Ythdf1^{-/-} mice bearing B16-OVA tumors was assessed. Although phagocytosis of tumor cells was similar in WT and Ythdf1^{-/-} mice (FIGS. 9B-9C), H-2K^{b}-SIINFEKL complexes were significantly higher in tumor-infiltrating Ythdf1^{-/-} DCs than in WT DCs (FIGS. 2G-2H). Furthermore, relative to splenic WT DCs, DCs from Ythdf1^{-/-} mice exhibited a higher potential for cross-presentation of soluble OVA in vitro (FIG. 9D). These data suggest that DCs from Ythdf1^{-/-} mice have improved antigen-presentation relative to DCs from WT mice.

### Example 6

To investigate whether the antitumor immunity relies on loss of Ythdf1 specifically in DCs, chimeric, DC-specific Ythdf1 knockout mice were generated. Specifically, irradiated mice were reconstituted with a 1:1 mixture of Ythdf1^{-/-} bone marrow cells and of WT bone marrow cells with a Zhtb46-DTR transgene, which drives expression of the diphtheria toxin receptor in classical DCs. Upon administration of diphtheria toxin (DT). WT cDCs expressing Zbtb46-DTR are selectively eliminated, with all remaining cDCs in Zbtb46-DTR:Ythdf1^{-/-} being Ythdf1-deficient. Chimeric Zbtb46-DTR:Ythdf1^{-/-} mice were also established as controls. It was found that B16-OVA tumors grew similarly in Zbtb46-DTR:Ythdf1^{+/+} and Zbtb46-DTR:Ythdf1^{-/-} chimeric mice that were not treated with DT (FIG. 2I). Importantly, treatment with DT substantially reduced tumor growth in Zbtb46-DTR:Ythdf1^{-/-} mice compared to Zbtb46-DTR:Ythdf1^{-/-} mice (FIG. 2I). These data demonstrate that loss of Ythdf1 specifically in cDCs is sufficient to generate the antitumor response. Together, these findings demonstrate that YTHDF1 in cDCs limits their cross-presentation capacity in vivo, and that altered T cell or DC homeostasis or development in Ythdf1^{-/-} mice does not substantially contribute to the antitumor activity.

### Example 7

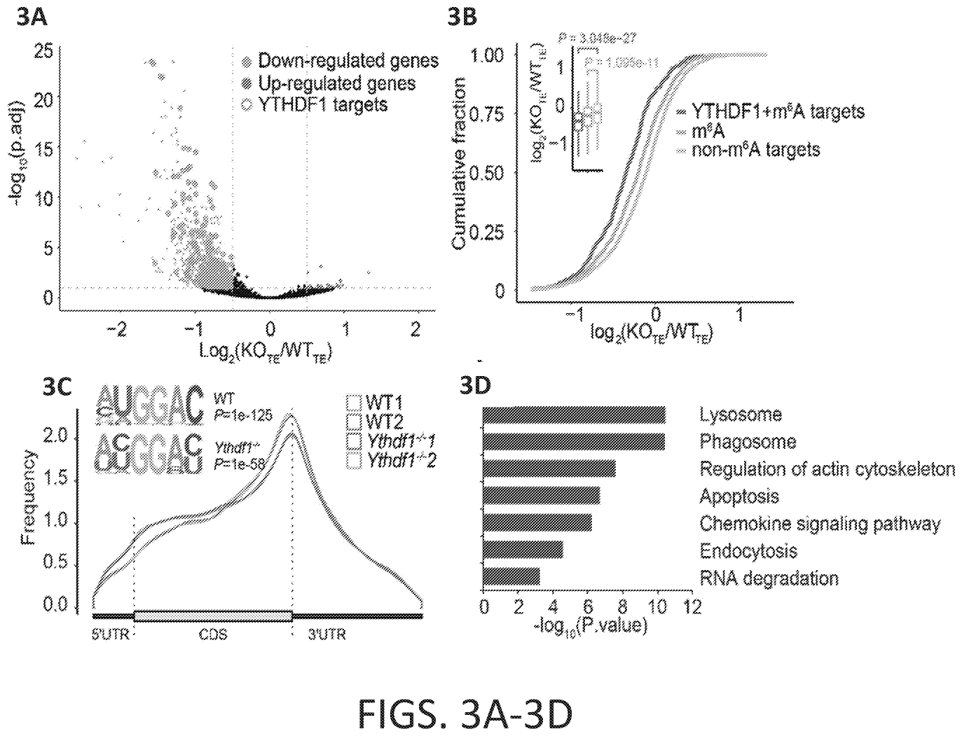

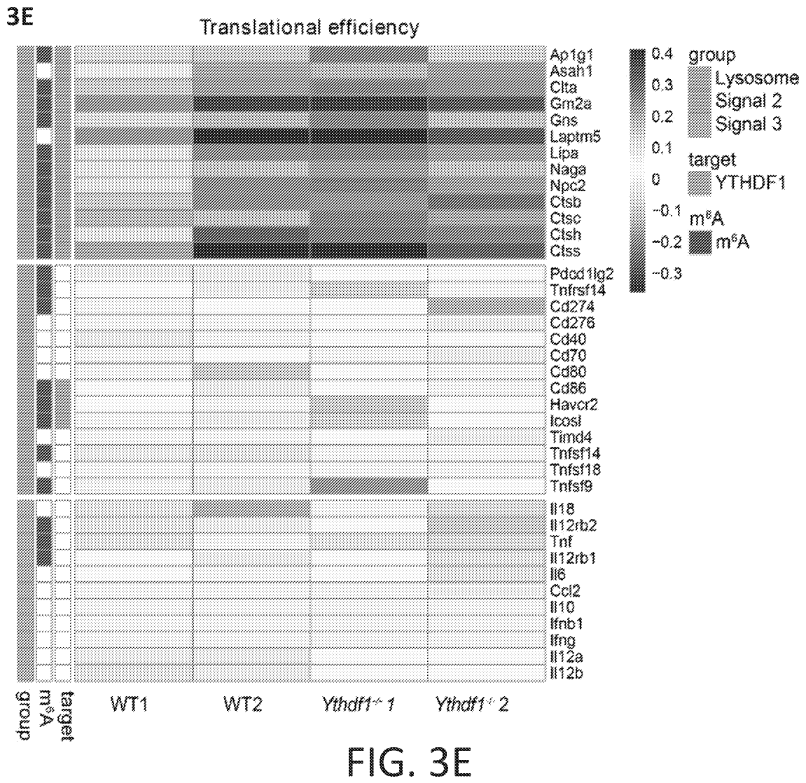

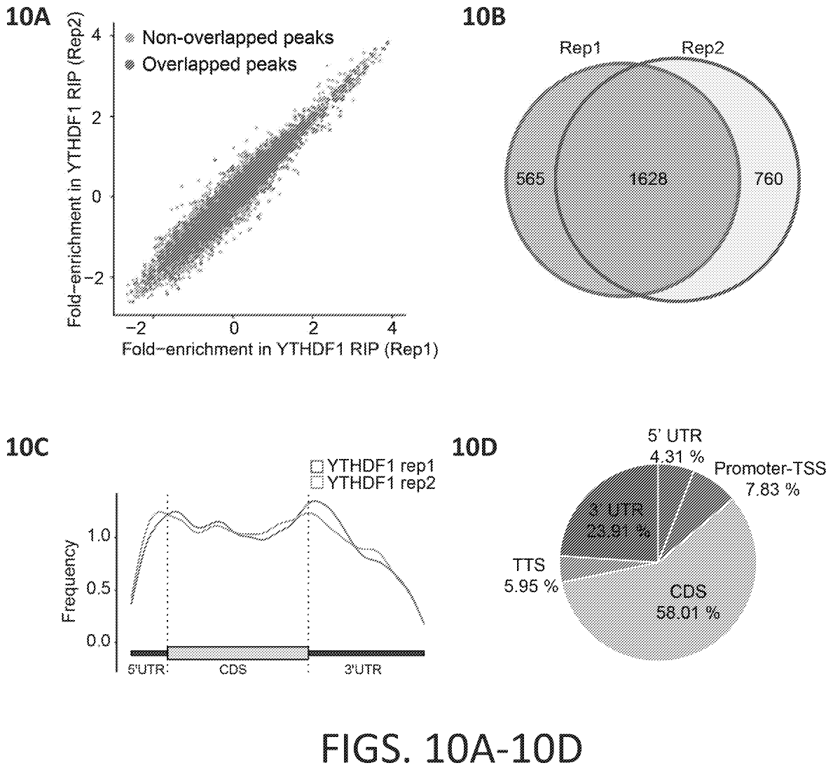

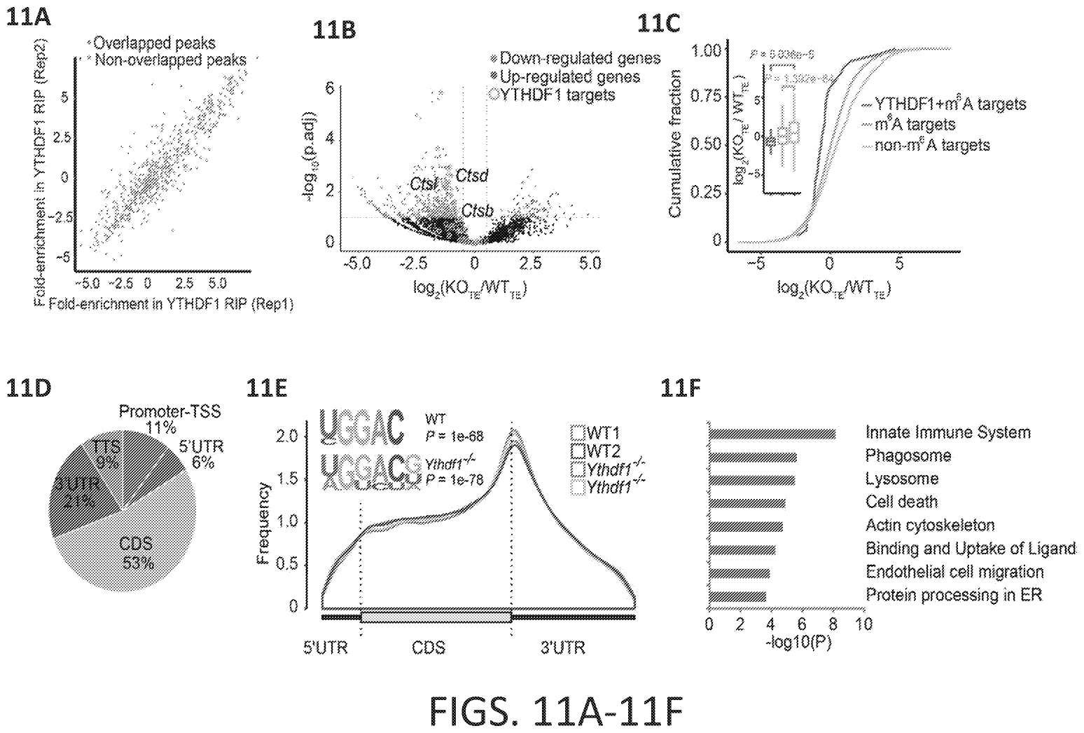

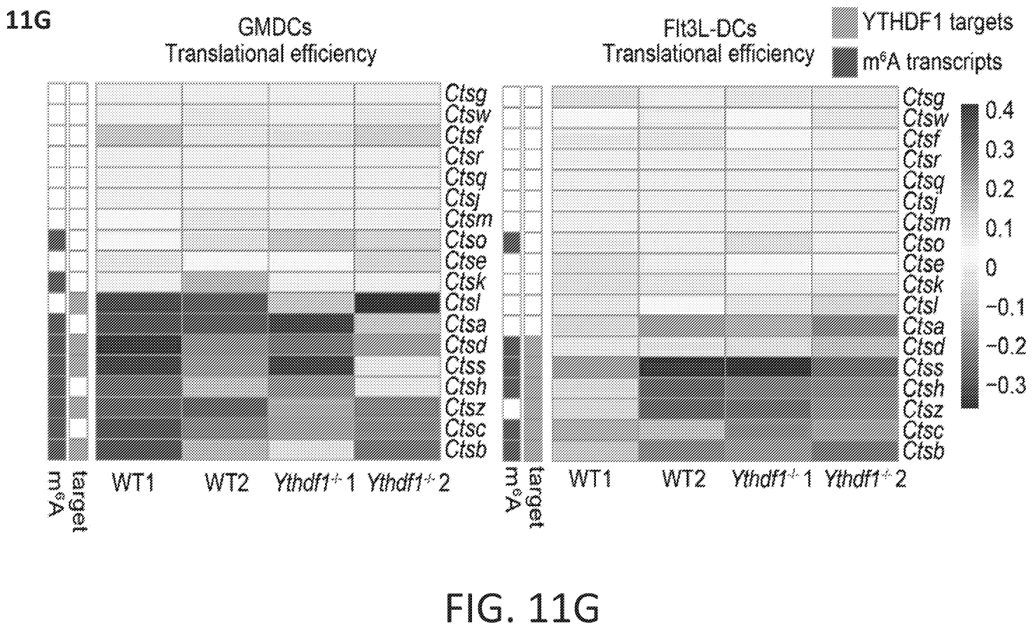

Next, how YTHDF1 deletion affects cross-presentation in DCs was investigated. RIP-Seq was performed to map target transcripts bound by YTHDF1 in Flt3L-DCs. YTHDF1-binding sites were highly reproducible between two biological replicates (FIGS. 10A-10B), and were predominantly distributed in the coding region and 3′ UTR (FIGS. 10C-10D). Given that YTHDF1 is known to affect mRNA translation, the translational efficiency of WT and Ythdf1^{-/-} DCs was assessed by ribosome profiling. Antibody-based m⁶A profiling and RNA-seq were also performed in the same cells. Transcripts were categorized into three groups: non-m⁶A marked transcripts, m⁶A-containing transcripts, and m⁶A-marked transcripts bound by YTHDF1. As expected, there was a significant decrease of translation efficiency, particularly for YTHDF1-targeted and m⁶A-marked transcripts, in Ythdf1^{-/-} DCs relative to WT DCs (FIGS. 3A-3B), while Ythdf1 deficiency did not substantially alter the distribution of m⁶A in mRNAs from DCs (FIG. 3C). To determine the functional pathways associated with YTHDF1-targeted mRNAs, m⁶A-marked mRNAs that are both targets of YTHDF1 and translationally regulated by YTHDF1 were investigated. Gene Ontology (GO) Enrichment Analysis was performed, which showed that YTHDF1-targeted transcripts were enriched for functions in the KEGG pathways of phagosome and lysosome (FIG. 3D). It was previously shown that limiting lysosomal proteolysis in DCs could enhance the cross-presentation by minimizing the destruction of internalized antigens. It was observed that the translation of a group of transcripts encoding lysosomal cathepsins, which are responsible for antigen degradation in DC lysosomes, was repressed in Ythdf1^{-/-} DCs compared with WT control (FIG. 3E). In contrast, the translational efficacy of co-stimulatory/inhibitory molecules (signal 2) and cytokines (signal 3) did not change markedly in YTHDF1-deficient Flt3L-DCs (FIG. 3E). In line with the observation in Flt3L-DCs, loss of Ythdf1^{-/-} resulted in the decreased translational efficiency of cathespins in GMCSF-induced bone marrow DCs (GMDCs) (FIGS. 11A-11F). In addition, it was found that multiple cathepsin transcripts are bound by the m⁶A reader protein YTHDF1, and knock-out of Ythdf1 resulted in a significant decrease of translation efficiency of these genes, in both GMDCs and Flt3L-DCs (FIG. 11G). These data suggest lysosomal cathepsins as the main targets controlled by YTHDF1, which subsequently affect the cross-priming capacity of DCs.

### Example 8

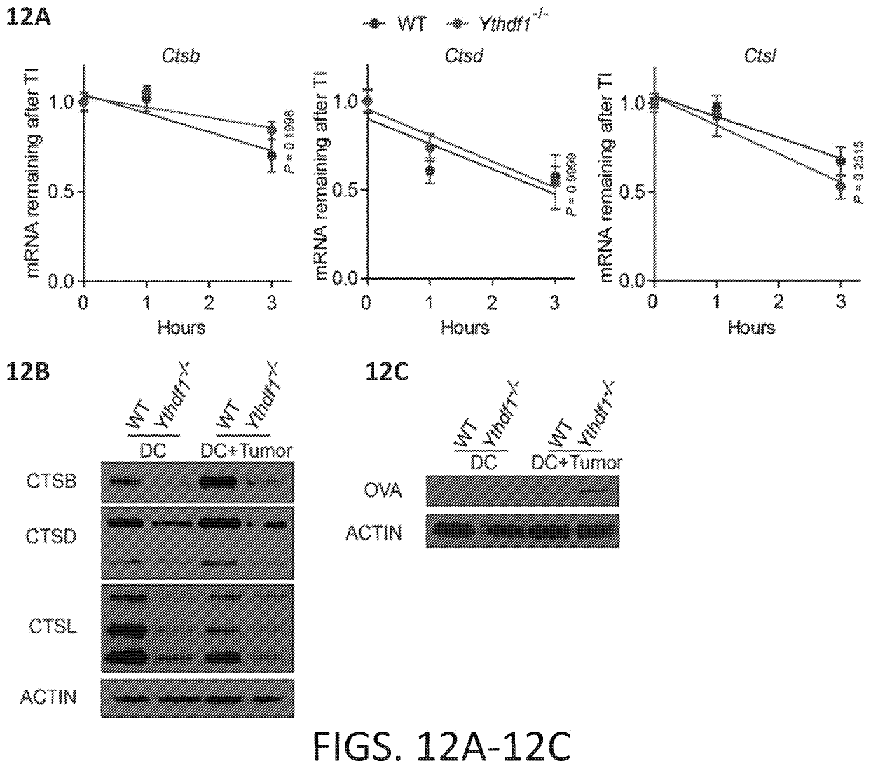

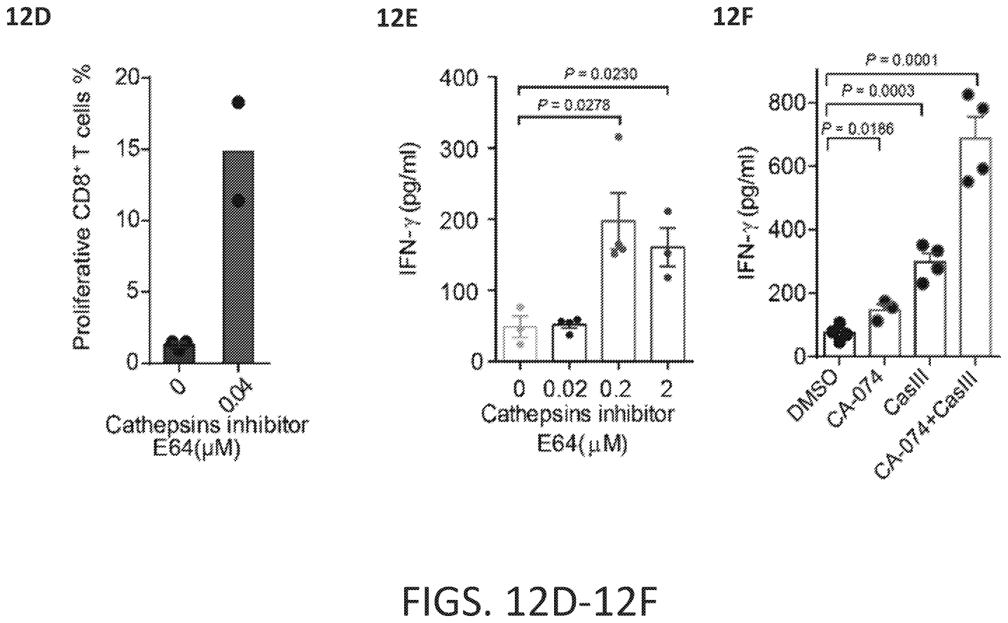

Next, ribosome-profiling results were confirmed by assessing the protein level of several cathepsins in splenic cDCs in vivo. Consistent with the observation that loss of Ythdf1 decreases the translational efficiency of m⁶A-marked transcripts, a similar downregulation of cathepsins was observed from Ythdf1^{-/-} cDCs in vivo and in vitro, but the decays of their transcripts were less affected (FIG. 4A; FIGS. 12A-12B). Whether reduced cathepsin levels may delay the degradation of the ingested neoantigens and facilitate a sustained antigen release within endosomes/lysosomes (e.g., contributing to the improved antigen presentation in Ythdf1^{-/-} DCs) was tested. To test this, GMDCs from WT and Ythdf1^{-/-} mice were co-cultured with B16-OVA cells overnight and assessed purified DCs for intact residual OVA. More residual intact OVA in Ythdf1^{-/-} DCs was observed compared to Ythdf1^{+/+} GMDCs (FIG. 12C). Whether the reduced translation efficiency of the YTHDF1 target cathepsins affects cross-presentation in Ythdf1^{-/-} DCs was subsequently investigated. It was found that inhibition of cathepsins using a broad-spectrum cysteine protease inhibitor E64 or more selective inhibitors CA-074 (for cathepsin B) and cathepsin L inhibitor III notably enhanced the efficiency of cross-priming in wild-type DCs (FIGS. 12D-12F). Moreover, the in vivo antitumor response was also markedly improved by cathepsin blockade (FIG. 4B), suggesting that cathepsins are critical factors determining the antitumor response in the current system. Collectively, these data show that loss of YTHDF1 in DCs attenuates antigen degradation by restricting the expression of lysosomal proteases, leading to improved cross-presentation and better cross-priming of CD8⁺ T cells.

### Example 9

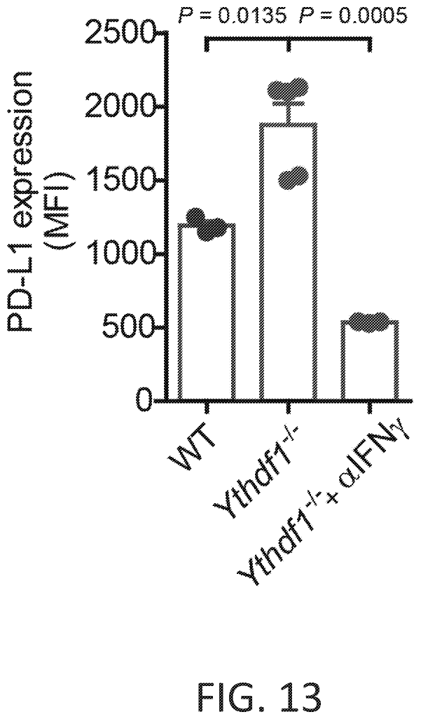

Whether loss of YTHDF1, with increased neoantigen-specific CD8⁺ T cells, might enhance the antitumor response of immune checkpoint blockade, which targets the T cell inhibitor receptor PD1, was investigated. Since Ythdf1^{-/-} mice showed a marked increase of IFNγ in CD8⁺ T cell and IFNγ signaling upregulates the expression of PD-L1, the ligand for PD1. PD-L1 levels were evaluated, and increased PD-L1 expression in tumor cells from Ythdf1^{-/-} tumor-bearing mice was observed (FIG. 4C), whereas neutralizing IFNγ diminished the expression of PD-L1 (FIG. 13). Whether PD-L1 blockade can potentiate the antitumor response in Ythdf1^{-/-} mice was then tested. WT and Ythdf1^{-/-} tumor-bearing mice were treated with an anti-PD-L1 antibody (clone 10F.9G2). Although tumor regression occurred in 40% of untreated Ythdf1^{-/-} mice or anti-PD-L1-treated WT mice, it was found that 100% of Ythdf1^{-/-} mice showed complete tumor regression after PD-L1 blockade (FIG. 4D). These data demonstrate that combining a checkpoint blockade with YTHDF1 depletion could be a potential new therapeutic strategy to improve the outcome in patients with low response to checkpoint blockade.

### 4. Materials and Methods

Mice.

Ythdf1^{-/-} mice were generated as previously described. Founder mice with mutant alleles were backcrossed to C57BL/6J for two generations. Mice used for experiments were further backcrossed to C57BL/6J for seven generations, totally 9 generations. To ensure the comparability in genetic background, mice were maintained by crossing heterozygous and heterozygous. Ythdf1^{-/-} mice or their littermates control WT mice were used in all experiments. Littermates were co-housing during experiments to reduce variants in their microbiome and environment. Primers used for genotyping of Ythdf1^{-/-} mice: CACCTGAGTTCAGATCATTAC (SEQ ID NO: 1) and GCTCCAGACTGTTCATCC (SEQ ID NO: 2). Female Rag2^{-/-} mice, 2C CD8⁺ T cell receptor (TCR)-Tg, CD11c-Cre and Zbtb46-DTR mice were purchased from Jackson laboratory. CD11c-CreMettl14^{f/f} conditional knockout mice were generated in house. All mice were used at 6-12 weeks of age. All the mice were maintained under specific pathogen-free conditions and used in accordance with the animal experimental guidelines set by the Institute of Animal Care and Use Committee. This study has been approved by the Institutional Animal Care and Use Committee of The University of Chicago.

Cell Lines.

MC38 is a murine colon adenocarcinoma cell line. MC38-zsGreen-OTIp (MC38-OZ) and B16-zsGreen-OTIp (B16-OZ) were selected for a single clone after being transduced by lentivirus expressing zsGreen-OTIp (SIINFEKEL). B16-OVA is an OVA-transfected clone derived from the murine melanoma cell line B16. MC38-SIY is an EGFR-SIY-transfected clone derived from the murine colon cell line MC38. Cells were maintained either in DMEM (Invitrogen) supplemented with 10% FBS and 1% penicillin-streptomycin supplemented with 2 mM L-glutamine. 1 mM sodium pyruvate and 0.1 mM non-essential amino acid at 37° C. in 5% CO2.

Primary Cell Cultures.

Single-cell suspensions of bone marrow cells were cultured in RPMI-1640 medium containing 10% fetal bovine serum, supplemented with 20 ng/ml GM-CSF (Biolegend). Fresh media with GM-CSF was added into culture on days 3 and 5. On day 6, CD11c⁺ DCs were purified using EasySep Mouse CD11c Positive Selection Kit II (STEMCELL). To culture Flt3L-DCs, single-cell suspensions of bone marrow cells were cultured in IMDM medium containing 10% fetal bovine serum at the concentration of 1×10⁶/ml. Cells were supplemented with 100 ng/ml Flt3-Ligand (PEPROTECH) for 9-10 days to obtain the Flt3L-DCs.

Tumor Growth and Treatments.

About 1×10⁶ B16-OVA or MC38 tumor cells were injected s.c. into the flank of mice. Tumor volumes were measured by length (a) and width (b) and calculated as tumor volume=ab²/2. Mice with tumor volumes less than 200 mm³ are considered to be survival. For in vivo depletion experiments, 200 μg of anti-CD8 antibody (clone YTS169.4) or anti-NK1.1 (clone PK136) was injected i.p. three days after tumor inoculation. To block cathepsins in vivo, mice were inoculated with 1×10⁶ B16-OVA cells. On day 11, mice with established tumors were treated with E64 intratumorally. For anti-PDL1 treatment, 1×10⁶ B16-OVA tumor cells were s.c. injected into the flank of mice. Tumors were allowed to grow for seven days and treated i.p. by anti-PDL1 (clone10F.9G2) or Rat Ig. Tumor-free mice after treatment were monitored over time and the percentage of tumor-regression was calculated. To block IFNγ, tumor-bearing mice were treated with 50 μg anti-IFNγ mAb (clone XMG1.2) intratumorally and PD-L1 expression on tumor cells was evaluated by flow cytometry. All antibodies were ‘InVivoMAb’ from BioXCell. For adoptive transfer of T cells. Rag mice were inoculated with 5×10⁵ B16-OVA on day 0. On the same day, T cells were purified from WT or Ythdf1^{-/-} mice using T cell negative isolation Kit (STEM CELL). 5×10⁶ T cells were intravenously (i.v.) injected into Rag2^{-/-} mice.

Generation of Bone Marrow Chimera.

To generate bone marrow chimeric mice, C57BL/6 mice were exposed to 800 rads of X-ray. After 24 hours, 5×10⁶ bone marrow cells. consisting of 2.5×10⁶ WT or Ythdf1^{-/-} BM cells and 2.5×10⁶ Zbtb46-DTR BM cells, were i.v. injected into irradiated mice. Six weeks after reconstitution, Zbtb46-DTR:Ythdf1^{+/+} and Zbtb46-DTR:Ythdf1^{-/-} mixed BM chimera mice were inoculated with 10⁶ B16-OVA cells and treated with 400 ng diphtheria toxin (sigma) or PBS every other day for sixteen days.

Flow Cytometry and Cell Sorting.

For flow cytometric analysis and cell sorting, tumors, lymph nodes and spleens were collected from mice and digested with 0.26 U/ml Liberase™ and 0.25 mg/ml DNase I at 37° C. for 30 min. Samples were then filtered through a 70 μm cell strainer and washed twice with staining buffer. Cells were re-suspended in staining buffer (PBS with 2% FCS and 0.5 M EDTA). Cells were incubated with Fc Block (clone 2.AG2; BioX Cell) for 10 min. Subsequently, specific antibodies were added and staining was continued for 30 min on ice. Information for all the antibodies used are provided in Table 1.

TABLE 1

| | | | | Catalog | Clone |
|---|---|---|---|---|---|
| Antibody | Application | Dilution fold | Company | number | number |
| Goat HRP-anti-GAPDH | WB | 1:10000 | ProteinTech | HRP-60004 | |
| Rabbit anti-YTHDF1 | IP&WB | 1:100 for IP, | ProteinTech | 17479-1-AP | |
| | | 1:1000 for WB | | | |
| Rabbit anti-YTHDF2 | WB | 1:1000 | ProteinTech | 24744-1-AP | |
| Anti-rabbit lgG-HRP | WB | 1:5000 | cell signaling | 7074S | |
| | | | technology | | |
| Mouse anti-YTHDF3 | WB | 1:500 | Santa-Cruz | sc-377119 | F-2 |
| Anti-mouse lgG-HRP | WB | 1:5000 | cell signaling | 7076S | |
| | | | technology | | |
| Rabbit anti-YTHDC1 | WB | 1:1000 | Abcam | ab122340 | |
| Goat anti-YTHDC2 | WB | 1:500 | Santa-Cruz | sc-249370 | G-19 |
| Anti-goat lgG-HRP | WB | 1:5000 | Abcam | ab6741 | |
| Anti-Chicken Egg | WB | 1:5000 | Sigma | C6534-2ML | |
| Albumin antibody | | | | | |
| produced in rabbit | | | | | |
| Mouse/Rat Cathepsin | WB | 1:1000 | R&D | AF1515 | |
| L Antibody | | | | | |
| β-Actin Antibody | WB | 1:2000 | Santa-Cruz | sc-47778 | |
| (C4) HRP | | | | HRP | |
| InVivoMAb anti-mouse | FcγR | 1:500 | BioX Cell | BE0307 | 2.4G2 |
| CD16/CD32 | blockade | | | | |
| CD11c Monoclonal | Flow | 1:500 | eBioscience | 25-0114-82 | N418 |
| Antibody (N418), | cytometry | | | | |
| PE-Cyanine7 | | | | | |
| PerCP/Cy5.5 | Flow | 1:500 | Biolegend | 101228 | M1/70 |
| Anti-Mouse/Human | cytometry | | | | |
| CD11b | | | | | |
| FITC Anti-Mouse | Flow | 1:500 | Biolegend | 128005 | HK1.4 |
| Ly-6C | cytometry | | | | |
| Brilliant Violet | Flow | 1:500 | Biolegend | 107635 | M5/114.15.2 |
| 510 ™ anti-mouse | cytometry | | | | |
| I-A/I-E | | | | | |
| AF 647 Anti- | Flow | 1:500 | eBioscience | 132121 | BM8 |
| Mouse F4/80 | cytometry | | | | |
| APC/Cy7 Anti- | Flow | 1:500 | Biolegend | 103116 | 30-F11 |
| Mouse CD45 | cytometry | | | | |
| Brilliant Violet | Flow | 1:500 | Biolegend | 101831 | M1/69 |
| 510 ™ anti-mouse | cytometry | | | | |
| CD24 | | | | | |
| PE Hamster Anti- | Flow | 1:500 | BD | 553769 | 16-10A1 |
| Mouse CD80 | cytometry | | Biosciences | | |
| PE/Cy5 Anti- | Flow | 1:500 | Biolegend | 105016 | GL-1 |
| Mouse CD86 | cytometry | | | | |
| PE anti-mouse | Flow | 1:500 | Biolegend | 124308 | 10F.9G2 |
| CD274 (B7-H1, | cytometry | | | | |
| PD-L1) | | | | | |
| PE-Cy7 Anti- | Flow | 1:500 | eBioscience | 25-0041-82 | GK1.5 |
| Mouse CD4 | cytometry | | | | |
| PE Anti-Mouse | Flow | 1:500 | Biolegend | 100708 | 53-6.7 |
| CD8a | cytometry | | | | |
| PE anti-mouse | Flow | 1:500 | Biolegend | 121612 | 1D4B |
| CD107a (LAMP-1) | cytometry | | | | |
| FITC Anti-Mouse | Flow | 1:500 | Biolegend | 101907 | 3C7 |
| CD25 | cytometry | | | | |
| FITC anti- | Flow | 1:501 | Biolegend | 103206 | RA3-6B2 |
| mouse/human | cytometry | | | | |
| CD45R/B220 | | | | | |
| PE anti- | Flow | 1:500 | Biolegend | 320008 | 150D |
| mouse/rat/human | cytometry | | | | |
| FOXP3 | | | | | |
| OVA257-264 | Flow | 1:500 | Thermofisher | 12-5743-82 | 25-D1.16 |
| (SIINFEKL) | cytometry | | | | |
| peptide bound to | | | | | |
| H-2Kb Monoclonal | | | | | |
| Antibody | | | | | |
| NK1.1 Monoclonal | Flow | 1:500 | Thermofisher | MM6628 | PK136 |
| Antibody (PK- | cytometry | | | | |
| 136), Pacific Blue | | | | | |
| iTAg Tetramer/PE- | Flow | 1:500 | MBL | TB-5001-1 | |
| H-2 Kb OVA | cytometry | | | | |
| (SIINFEKL) | | | | | |
| Cathepsin A | Flow | 1:200 | Proteintech | 15020-1-AP | |
| Rabbit polyAb | cytometry | | | | |
| Cathepsin H | Flow | 1:200 | Proteintech | 10315-1-AP | |
| Rabbit polyAb | cytometry | | | | |
| Cathepsin B | Flow | 1:300 for flow | cell signaling | 31718S | D1C7Y |
| (D1C7Y) | cytometry & | cytometry, | technology | | |
| Rabbit mAb | WB | 1:1000 for WB | | | |
| Rb mAb to | Flow | 1:300 for flow | Abcam | ab75852 | EPR3057Y |
| Cathepsin D | cytometry & | cytometry, | | | |
| | WB | 1:1000 for WB | | | |
| Alexa Fluor 568 | Flow | 1:500 | ThermoFisher | A-11036 | |
| Goat-anti-Rabbit | cytometry | | | | |
| IgG | | | | | |
| InVivoMAb anti- | In vivo | 200 μg | BioX Cell | BE0036 | PK136 |
| mouse NK1.1 | depletion | | | | |
| InVivoMAb anti- | In vivo | 200 μg | BioX Cell | BE0117 | YTS169.4 |
| mouse CD8α | depletion | | | | |
| InVivoMAb anti- | In vivo | 50 μg | BioX Cell | BE0055 | XMG1.2 |
| mouse IFNγ | blockade | | | | |
| anti-IFNγ | ELISPOT | 1:250 | BD | 554408 | XMG1.3 |
| antibody | | | Biosciences | | |
| CD8 | IHC | 1:400 | DAKO | M7103 | C8/144B |

OT-I specific T cells were stained using iTAg Tetramer/H-2K^{b}OVA (SIINFEKEL) (MBL). After a washing step, cells were either analyzed on a BD Fortessa (BD) or sorted by AriaIIIu (BD). For the staining of cathepsins, splenocytes were stained with CD11c. B220, MHCII, CD8 and CD11b and then fixed with 4% PFA (Biolegend) for 30 minutes. Fixed cells were then washed twice with the 1× intracellular staining perm and wash buffer (Biolegend). Antibody against CTSA, CTSB, CTSD or CTSH was added and incubated overnight respectively. Alexa Fluor 568 Goat-anti-Rabbit IgG was added as the secondary antibody. CD11c⁺MHCII⁺B220⁻ was gated and the expression of cathepsins was evaluated by the fluorescence intensity. Analysis of flow cytometry data was performed using Flowjo (Treestar).

Measurement of IFNγ-Secreting CD8⁺ T Cells by ELISPOT Assay.

For antigen-specific CD8⁺ T cell functional assay in the B16-OVA model, 12 days after tumor inoculation, 3×10⁵ lymphocytes were re-stimulated with 1 μg/ml SIINFEKEL or MC38 tumor cells (lymphocyte:MC38=50:1) for 48 hours. 96-well HTS-IP plate (Millipore) was pre-coated with anti-IFNγ antibody (BD Bioscience) with a 1:250 dilution overnight at 4° C. After co-culture, cells were removed. About 2 mg/mil biotinylated anti-IFNγ antibody (BD Bioscience) with a 1:250 dilution was added and incubated for 2 h at room temperature or overnight at 4° C. Avidin-horseradish peroxidase (BD Bioscience) with a 1:1000 dilution was then added and the plate was incubated for 1 h at room temperature. The cytokine spots of IFN-γ were developed according to product protocol (BD Bioscience).

Antigen-Presentation Assay.

For cross-presentation of tumor neoantigen, CD11b⁺ or CD8⁺ DC were purified from draining lymph node of WT or Ythdf1^{-/-} mice six days after inoculating with B16-OVA, MC38-OTIp or MC38-EGFR-SIY. OT-I or 2C naive CD8⁺ T cells were isolated from lymph nodes and spleen of 6 to 12-week-old mice. Negative selection was carried out with a negative CD8 isolation kit (StemCell Technologies, Inc.) following manufacturer's instruction. DCs were co-cultured with OT-1 naive CD8 T cells at the ratio of 1:10 for three days with or without 1 μg/ml SIINFEKEL peptide. For cross-presentation of soluble OVA, splenic DCs were sorted and stimulated with 100 ng/ml LPS overnight. DCs were then pulsed with different concentration of OVA (endotoxin free, Sigma) for 5 hours. Cells were washed and co-cultured with OT-I naive CD8⁺ T cells for three days. For in vitro cross-presentation of tumor neoantigen, Flt3L-DCs were collected on day 9-10 and co-cultured with necrotic B16-OVA tumor cells overnight. B220⁻CD11c⁺ cells were subsequently purified. GMDCs from Mettl14^{f/f} or CD11c-CreMettl14^{f/f} mice were harvested on day 6 and co-cultured with necrotic B16-OVA tumor cells for 16 hours. To inhibit cathepsins, GMDCs were pre-treated with E64 (sigma) for two hours followed by co-culturing with tumor cells. CD11c⁺ cells were then purified and incubated with naive CD8⁺ T cells from OT-I mice for three days. IFN-T production was detected by IFN-γ Flex Set CBA assay (BD Bioscience). To inhibit cathepsins in ex vivo cDCs, WT or Ythdf1^{-/-} mice were inoculated with 5×10⁵ MC38. 36 hours after tumor inoculation, spleens were collected and digested, and CD11c⁺ DCs were purified using EasySep Mouse CD11c Positive Selection Kit 11(STEMCELL). CD11c⁺ DCs were then treated with 0.04 μM E64 (Sigma) overnight followed by co-culturing with OVA protein for 4 hours. Any free OVA protein was then removed from the culture medium, and CD11c⁺ cells were incubated with CTV-labelled OT-1 cells for three days. The cross-priming capacity of DC was analyzed by the dilution of CTV in CD8⁺ T cells. For cathepsins inhibition assay in vitro, Flt3L-DCs were treated with 5 μg/ml CA-074 methyl ester (Selleck), 5 μg/ml cathepsin L inhibitor III(Sigma) or the combination (5 μg/ml CA-074 methyl ester and 5 μg/ml cathepsin L inhibitor III) for 2 h followed by co-culturing with necrotic B16-OVA for 16 h, and then Flt3L-DCs were purified using EasySep Mouse CD11c Positive Selection Kit II (STEMCELL). The purified cells were incubated with OT I cells at the ratio of 1:20 for three days. The cross-priming capacity of DCs was then evaluated by the IFN-γ production. To detect the MHC-H2K^{b}-SIINFEKEL, mice were inoculated with B16-OVA. After 12 days, tumors were collected and tumor infiltrating DCs (CD45⁺CD11b⁺ly6c⁻MHCII⁺CD24⁺CD11c⁺) were stained with monoclonal antibody 25.D1.

Cell Trace Violet Labelling.

10 million splenocytes from naive OT-I mouse were re-suspended in 1 ml PBS followed by incubating with 5 uM CellTrace™ Violet Dye (CTV, ThermoFisher) at 37° C. for 20 minutes. 5 ml RPMI-1640 medium was added to the cells and incubated for 5 minutes to remove the free dye in the solution. These cells were then centrifuged and incubated with pre-warmed RPMI—for at least 10 minutes at room temperature for subsequent analysis.

Rip-Seq.

About 20 million GMDCs were harvested and co-cultured with or without necrotic B16-OVA overnight. The procedure was adapted from the previous report. Five million Flt3L-DCs were harvested. DCs were then purified and pelleted by centrifuge for 5 min. Cells were washed twice with cold PBS and the cell pellet was re-suspended with 2 volumes of lysis buffer (150 mM KCl. 10 mM HEPES pH 7.6, 2 mM EDTA. 0.5% NP-40.0.5 mM DTT, 1:100 protease inhibitor cocktail, 400 U/mL RNase inhibitor). Lysate was incubated on ice for 5 min and centrifuged for 15 min to clear the lysate. About 1/10 volume of cell lysate was saved as input and total RNA was extracted by Trizol. The rest of cell lysate was incubated with 5 μg anti-YTHDF1 (Proteintech) at 4° C. overnight with gentle rotation followed by incubation with 40 μl protein G beads for 1 hour at 4° C. The beads were then washed five times with 1 mL ice-cold washing buffer (200 mM NaCl. 50 mM HEPES pH 7.6, 2 mM EDTA, 0.05% NP-40, 0.5 mM DTT. 200 U/mL RNase inhibitor). The IP complex was resuspended in 400 μl 1×Proteinase K and digested with 2 mg Proteinase K at 55° C. for 1 hour. RNA was then extracted by RNA isolation kit (Zymo). Input and IP RNA of each sample were used to generate the library using TruSeq stranded mRNA sample preparation kit (Illumina).

m⁶A-Seq.

Total RNA was isolated from DCs. Polyadenylated RNA was further enriched from total RNA by using Dynabeads® mRNA Purification Kit (Invitrogen). RNA samples were fragmented into ˜100-nucleotide-long fragments with sonication. Fragmented RNA (100 ng mRNA or 5 μg total RNA) was performed m⁶A-IP following EpiMark N6-methyladenosine enrichment kit (NEB E1610S) protocol. RNA was enriched through RNA Clean&Concentration-5 (Zymo Research) and used for library generation with SMARTer Stranded Total RNA-Seq Kit (Takara). Sequencing was performed at the University of Chicago Genomics Facility on an Illumina HiSeq4000 machine in single-read mode with 50 bp per read. Sequencing reads were aligned to the mouse genome mm9 by STAR (version 2.6.0c). The m⁶A-enriched regions (peaks) in each m⁶A-IP sample were detected by MACS2 (version 2.1.1.20160309) with q value less than 0.01 and corresponding m⁶A-Input sample was used as the control. Peaks that were detected by both replicates were considered as high confident peaks. The peaks annotation and binding motif were analyzed by HOMER (version 4.9).

Ribosome Profiling.

About 5×10⁶ DCs were treated with 100 μg/ml cycloheximide (CHX) for 7 minutes. The cells were then harvested by the cell lifter. The cell suspension was spun at 400 g for 5 min and the cell pellet was washed twice with 5 ml cold PBS with CHX (100 μg/ml). About 200 μl lysis buffer (10 mM Tris, pH 7.4, 150 mM KCl, 5 mM MgCl₂, 100 μg/ml CHX, 0.5% Triton-X-100, freshly added 1:100 protease inhibitor, 40 U/ml SUPERasin) was added to the cell pellet and lysed on ice for 15 minutes with rotating. About 10% clarified lysate was saved as INPUT and the rest lysate was separated through a 5 ml 10%-50% sucrose gradient and centrifuged at 4C for 2 h at 28,000 r.p.m. Fractions were collected separately and analyzed by Qubit™ RNA HS Assay Kit (Invitrogen). The fractions corresponding to monosome or polysome were combined respectively and concentrated on Amicon-Ultra 100K columns (Millipore). Two A260 units of ribosome fractions were digested with 60 U RNase 1 (Ambion) at room temperature for 30 minutes. RNA was extracted by RNA Clean&Concentrate (Zymo) and ribosomal RNA were deleted prior to size selection. RNA fragments (26-32 nt) were isolated by 15% denaturing Urea-PAGE gel. RNA was eluted from gel in elution buffer (300 mM sodium acetate pH 5.2, 1 mM EDTA) followed by phenol-chloroform extract and ethanol precipitation. RNA fragments were dephosphorylated and prepared into libraries by SMARTer® smRNA-Seq Kit (Clontech). The first 3 bases of sequencing reads were removed fastx_trimmer (version 0.0.14). The adapter sequences and polyA tails were firstly trimmed from sequencing reads by using cutadapt (version 1.15) with—minimum-length 18-n 3-a “AAAAAAAAAAAAAA”-a “AGATCGGAAGAGCACACGTCTGAACTCCAGTCAC” (SEQ ID NO: 3) parameters. Trimmed reads were filtered for mitochondrial DNA and ribosomal RNA by Bowtie2 (version 2.3.4). All remaining reads were mapped to the mouse genome mm9 with STAR (version 2.6.0c). Uniquely mapped reads were selected by using SAMtools (version 1.7) with mapping quality >=20, and then removing duplication. The raw counts of coding regions were calculated by HOMER (version 4.9). The differentially TE (translational efficiency, Equation 1) genes were detected by Bioconductor DESeq2 package (version 1.18.1) with p.adj<=0.1 and |log 2FoldChange|>=0.5.

$$TE = \frac{\frac{{Ythdf}_{1}^{- \text{/} -} ( ribosome )}{{Ythdf}_{1}^{- \text{/} -} ( RNAseq )}}{\frac{WildType ( ribosome )}{WildType ( RNAseq )}} \qquad ( Equation 1 )$$

Measurement of RNA Lifetime.

DCs were seeded in 24-well plate at 50% confluency. After 2 h, actinomycin D was added to 5 mg/ml at 3 h. 1 h and 0 h before collection. The total RNA was purified by RNeasy kit with an additional DNase-I digestion step on column. RNA quantities were determined by RT-qPCR. The specific primers used are as follows: Ctsb_Forward: CTGCTTACCATACACCAT (SEQ ID NO: 4); Ctsb_Reverse: TCCTTCACACTGTTAGAC (SEQ ID NO: 5); Ctsd_Forward: GGCAAGAGGTATCAAGGT (SEQ ID NO: 6); Ctsd_Reverse: CAGGTAGAAGGAGAAGATGT (SEQ ID NO: 7); Ctsl_Forward: GAGTTCGCTGTGGCTAAT (SEQ ID NO: 8); Ctsl_Reverse: GAGGTTCTTGCTGCTACA (SEQ ID NO: 9); Gapdh_Forward: ACCTGCCAAGTATGATGA (SEQ ID NO: 10); Gapdh_Reverse: GGAGTTGCTGTTGAAGTC (SEQ ID NO: 11).

Immunohistochemistry of Human Biopsies.

All samples encompassing tumor biopsies from 22 colorectal cancer patients were obtained with informed consent under a protocol approved by the University of Chicago Institutional Review Board. Information about the patient sex, age, and tumor characteristics are given in Table 2.

TABLE 2

| Sample | | | Tumor | Histologic | |
|---|---|---|---|---|---|
| ID | Gender | Age | Location | type of tumor | Pathologic Staging (pTNM) |
| 1 | Female | 48.3 | Proximal/Right | Adenocarcinoma | pT3, N1a, Mx |
| | | | Colon | | |
| 2 | Female | 47.8 | Proximal/Right | Adenocarcinoma | pT4b (tumor directly invades |
| | | | Colon | | or is adherent to other); pN0: |
| | | | | | No regional lymph node |
| | | | | | metastasis |
| 3 | Female | 59.4 | Proximal/Right | Adenocarcinoma | pT3 (tumor invades into |
| | | | Colon | | pericolic tissues); pN0 |
| | | | | | (no regional lymph node |
| | | | | | metastasis) |
| 4 | Male | 68.5 | Proximal/Right | Adenocarcinoma | ypT4b, N0 (No regional |
| | | | Colon | | lymph node metastasis), MX, |
| | | | | | R1 |
| 5 | Male | 62.9 | Distal/Left | Adenocarcinoma | pT2(tumor invades |
| | | | Colon | | muscularis propria), N0, Mx a |
| 6 | Female | 74.1 | Proximal/Right | Adenocarcinoma | pT2(Tumor invades |
| | | | Colon | | muscularis propria), N0, Mx = |
| | | | | | not specified |
| 7 | Female | 46.2 | Distal/Left | Adenocarcinoma | pT3, N0, pM = not applicable |
| | | | Colon | | |
| 8 | Male | 65.9 | Proximal/Right | Adenocarcinoma | pT2, N0, pM = not applicable |
| | | | Colon | | |
| 9 | Female | 52.6 | Distal/Left | adenocarcinoma | pT2, N1a |
| | | | Colon | | |
| 10 | Male | 60.6 | Proximal/Right | adenocarcinoma | pT3 (m), N2a |
| | | | Colon | | |
| 11 | Male | 46.9 | Distal/Left | adenocarcinoma | ypT3N2a |
| | | | Colon | | |
| 12 | Male | 79.6 | Proximal/Right | adenocarcinoma | pT2, N1b, Distant Metastasis |
| | | | Colon | | (pM): Not applicable |
| 13 | Female | 42.1 | Proximal/Right | adenocarcinoma | ypT3, N1, M1 |
| | | | Colon | | |
| 14 | Male | 79.4 | Distal/Left | adenocarcinoma | pT3, N0 |
| | | | Colon | | |
| 15 | Female | 75.7 | Distal/Left | adenocarcinoma | pT2, N0, pM = not applicable |
| | | | Colon | | |
| 16 | Male | 65.6 | Distal/Left | adenocarcinoma | pT3(m), N0, Mx |
| | | | Colon | | |
| 17 | Male | 55.1 | Distal/Left | adenocarcinoma | pT1, N0 |
| | | | Colon | | |
| 18 | Female | 66.7 | Proximal/Right | adenocarcinoma | pT4a, pN2b, pM1b |
| | | | Colon | | |
| 19 | Male | 86.8 | Distal/Left | adenocarcinoma | pT3, pN2a, pM: Not applicable |
| | | | Colon | | |
| 20 | Male | 81 | Distal/Left | adenocarcinoma | pT3N0 |
| | | | Colon | | |
| 21 | Male | 77.3 | Proximal/Right | adenocarcinoma | pT4b, N0, Distant Metastasis |
| | | | Colon | | (pM): Not applicable |
| 22 | Male | 68.2 | Distal/Left | adenocarcinoma | pT3, N2a, Distant Metastasis |
| | | | Colon | | (pM): Not applicable |

To stain YTHDF1 and CD8, antigen retrieval was performed with 10 mM Tris base, 1 mM EDTA, 0.05% Tween 20, pH9. Slides were processed with the VECTASTAIN Elite ABC HRP kit and DAB Substrate Kit (Vector Laboratories). Slides were counterstained with hematoxylin and dehydrated through graded alcohols and xylene. A total of 22 tumor samples had sufficient tissue for unambiguous analyses. For THC quantification, DAB stains of IHC images were separated by color deconvolution algorithms47 in Fiji, aderivative of ImageJ. The mean DAB intensity of 3 random images at 795×650 pixels was calculated and converted into optical density (OD). CD8 positive cells were analyzed by Image J cell counter. The average infiltration of CD8⁺ cell and average expression of YTHDF1 were assessed within the surrounding stroma tissues.

Western Blot Analysis.

To detect the expression of cathepins, GMDCs were harvested on day 6 and co-cultured with necrotic B16-OVA cells at the ratio of 1:1 for 16 hours. CD11c⁺ DCs were then purified. Equal numbers of cells were lysed on ice for 15 mini using 1×lysis buffer (CST) supplemented with a protease inhibitor cocktail (Calbiochem). Cell lysis was centrifuged at 16,100 g at 4° C. for 15 min. Clarified supernatant was loaded into 4-12% NuPAGE Bis-Tris gel and transferred to PVDF membranes (Life Technologies). Membranes were blocked for 1 hour in 5% milk TBST and then incubated with primary antibodies in the blocking buffer overnight at 4° C. After 5 times washing, membranes were incubated with secondary antibodies for 1 hour at room temperature. The information for all the antibodies used are provided in Table 1.

Degranulation of Tumor Infiltrating NK Cells.

Tumor infiltrating leukocytes were resuspended at 5×10⁵/ml and stimulated with phorbol-12-myristate-13-acetate (PMA) (2.5 μg/ml) and ionomycin (0.5 μg/ml) in 96-well plate. CD107α-PE antibody and 1×bredeldin A (Biolegend) were added directly to the well and incubated for 4 h at 37° C. in 5% CO2. Cells were stained for CD45 and NKL1.1 (BD Biosciences) for 30 min. Samples were washed and then fixed in 1% paraformaldehyde.

Phagocytosis In Vivo.

About 5×10⁵ B16F10 cells expressing zsGreen-OTI were injected s.c. into WT and Ythdf1^{-/-} mice. Tumor tissues were harvested and digested.

Maturation of DC.

GMDCs were harvested and co-cultured with 100 ng/ml LPS overnight. The cytokine production was measured by mouse inflammation kit (BD).

Identification of Off-Target Site and T7EI Assay.

Identified two off-target loci for each sgRNA site with highest score were selectively amplified by primers listed in Table 2. HiPure Tissue DNA Mini Kit (Magen, D3121-03) was used to extract genomic DNA from the tails of WT and Ythdf1^{-/-} a mice. The PCR reactions to amplify 350 bp fragment (for Y1 mouse) and 510 bp fragment (for WT mouse) were carried out in 30 μl reaction, using 15 μl of 2× EasyTaq PCR SuperMix (AS111 TransGen Biotech). 0.75 μM each of forward and reverse primers and 1 μl genomic DNA. The reaction products were subjected to 1.5% agarose gel electrophoresis. For T7E1 cleavage assay, equal volume PCR products of Ythdf1^{-/-} and WT mouse were mixed and then denatured and annealed in NEBuffer 2 (NEB) using a thermal cycler. Hybridized PCR products were digested with T7 endonuclease I (NEB, M0302L) or ddH2O (as control) for 20 minutes at 37° C. and subjected to 1.5% agarose gel electrophoresis.

Statistical Analysis and Reproducibility.

No statistical method was used to predetermine sample size. Mice were assigned at random to treatment groups for all mouse studies and, where possible, mixed among cages. There were no mice excluded from experiments. Blinded staining and blinded analysis were performed for IHC experiment. Experiments were independently repeated two to three times. Data were analyzed using Prism 5.0 software (GraphPad) and presented as mean values±s.e.m. The P values were assessed using one-tailed or two-tailed unpaired Student's t-test. For survival curve, statistics were done with the log-rank (Mantel-Cox) test. For translational efficiency. P values were calculated by likelihood ratio test and adjusted by Benjamini & Hochberg method. For cumulative distribution, two-sided Kolmogorov-Smirnov test was used to calculate the P values.

Data Processing and Analysis.

Illumina reads were post-processed and aligned to the mouse mm9 assembly using STAR program (version 2.6.0c) with default parameters. To visualize sequencing signals in the genome browser, RIP-seq and msA-seq bigwig files were generated with bamCoverage function from deepTools (version 3.0.1) with ‘-bs=1—normalizeUsing BPM’. For RIP-seq, Piranha software (version 1.2.1) was to detect the binding sites of YTHDF1 with “-b 100-i 100”. Metagene plots were performed by the Bioconductor GUITAR package (version 1.16.0). Peaks that were detected by both replicates were considered as high confident peaks. GO term analyses were performed by metascape.

Data Availability.

The data that support the findings of the present disclosure are available from the corresponding author upon reasonable request. RIP-seq, Ribo-seq and m⁶A-seq data sets have been deposited in Gene Expression Omnibus under the accession number GSE115106. A summary of sequencing experiments is provided in Table 3. The differential translational efficiency results can be made available upon request.

TABLE 3

| | | | | | |
|---|---|---|---|---|---|
| Sample | rawReads | mappedReads | mapRatio | UniqueReads | UniqueRatio |

Summary statistics for MeRIP sequencing experiment.
Flt3L-DCs

| | | | | | |
|---|---|---|---|---|---|
| MeRIP_wt_input1 | 22066802 | 20187629 | 0.91 | 19524641 | 0.97 |
| MeRIP_wt_input2 | 18896973 | 17126164 | 0.91 | 16565325 | 0.97 |
| MeRIP_ko_input1 | 12499957 | 10441974 | 0.84 | 9882494 | 0.95 |
| MeRIP_ko_input2 | 6178443 | 5086508 | 0.82 | 4829848 | 0.95 |
| MeRIP_wt_IP1 | 18914882 | 17406075 | 0.92 | 16885629 | 0.97 |
| MeRIP_wt_IP2 | 14850481 | 13637258 | 0.92 | 13225871 | 0.97 |
| MeRIP_ko_IP1 | 27192835 | 23529702 | 0.87 | 22625370 | 0.96 |
| MeRIP_ko_IP2 | 13972821 | 11987963 | 0.86 | 11512812 | 0.96 |

GMDCs

| | | | | | |
|---|---|---|---|---|---|
| MeRIP_wt_input1 | 28551565 | 26514705 | 0.93 | 19204549 | 0.72 |
| MeRIP_wt_input2 | 28528986 | 26516721 | 0.93 | 18936050 | 0.71 |
| MeRIP_ko_input1 | 29030942 | 26613287 | 0.92 | 19307132 | 0.73 |
| MeRIP_ko_input2 | 27169760 | 24956116 | 0.92 | 18167243 | 0.73 |
| MeRIP_wt_IP1 | 28291093 | 27748060 | 0.98 | 24896670 | 0.90 |
| MeRIP_wt_IP2 | 34180741 | 33535024 | 0.98 | 30095313 | 0.90 |
| MeRIP_ko_IP1 | 22708693 | 22151135 | 0.98 | 19858221 | 0.90 |
| MeRIP_ko_IP2 | 26732331 | 26048436 | 0.97 | 23372187 | 0.90 |

Summary statistics for Ythdf1-RIP sequencing experiment.
Flt3L-DCs

| | | | | | |
|---|---|---|---|---|---|
| Ythdf1_RIP_input1 | 23419684 | 22503004 | 0.96 | 21831779 | 0.97 |
| Ythdf1_RIP_input1 | 30933222 | 29772784 | 0.96 | 28887892 | 0.97 |
| Ythdf1_RIP_IP1 | 17808787 | 11689651 | 0.66 | 11215830 | 0.96 |
| Ythdf1_RIP_IP2 | 14697186 | 9577188 | 0.65 | 9189335 | 0.96 |

GMDCs

| | | | | | |
|---|---|---|---|---|---|
| Ythdf1_RIP_input1 | 15929824 | 15520067 | 0.97 | 13443554 | 0.87 |
| Ythdf1_RIP_input1 | 14831765 | 14546176 | 0.98 | 12629905 | 0.87 |
| Ythdf1_RIP_IP1 | 11578432 | 8250988 | 0.71 | 8028954 | 0.97 |
| Ythdf1_RIP_IP2 | 10779780 | 7371189 | 0.68 | 7178380 | 0.97 |

Summary statistics for Ribo sequencing experiment.
Flt3L-DCs

| | | | | | |
|---|---|---|---|---|---|
| Ribo_wt_input1 | 14353853 | 14000072 | 0.98 | 9840118 | 0.70 |
| Ribo_wt_input2 | 16978427 | 16560575 | 0.98 | 11572726 | 0.70 |
| Ribo_ko_input1 | 7997730 | 7400355 | 0.93 | 4650859 | 0.63 |
| Ribo_ko_input2 | 8515757 | 7813383 | 0.92 | 4754357 | 0.61 |
| Ribo_wt_ip1 | 13096471 | 9563047 | 0.73 | 3926799 | 0.41 |
| Ribo_wt_ip2 | 9591022 | 7259191 | 0.76 | 2949562 | 0.41 |
| Ribo_ko_ip1 | 17761548 | 14796902 | 0.83 | 5634634 | 0.38 |
| Ribo_ko_ip2 | 16482288 | 13272302 | 0.81 | 5076220 | 0.38 |

GMDCs

| | | | | | |
|---|---|---|---|---|---|
| Ribo_wt_input1 | 12475501 | 9899575 | 0.79 | 4213730 | 0.43 |
| Ribo_wt_input2 | 11953956 | 9555210 | 0.80 | 4051006 | 0.42 |
| Ribo_ko_input1 | 10430201 | 8379582 | 0.80 | 3771784 | 0.45 |
| Ribo_ko_input2 | 13114316 | 10588500 | 0.81 | 4699991 | 0.44 |
| wt_monosome1 | 10968673 | 6606682 | 0.60 | 2408519 | 0.36 |
| wt_monosome2 | 10725649 | 4128146 | 0.38 | 1865605 | 0.45 |
| ko_monosome1 | 6340117 | 2504585 | 0.40 | 967011 | 0.39 |
| ko_monosome2 | 7607352 | 3063659 | 0.40 | 1496955 | 0.49 |
| wt_polysome1 | 5970349 | 2371023 | 0.40 | 865580 | 0.37 |
| wt_polysome2 | 6197195 | 2356148 | 0.38 | 1085431 | 0.46 |
| ko_polysome1 | 9760993 | 2883708 | 0.30 | 1090299 | 0.38 |
| ko_polysome2 | 7984928 | 2758005 | 0.35 | 963418 | 0.35 |

Summary statistics for DC data sequencing experiment.
Flt3L-DCs

| | | | | | |
|---|---|---|---|---|---|
| MeRIP_wt_input1 | 22066802 | 20187629 | 0.91 | 19524641 | 0.97 |
| MeRIP_wt_input2 | 18896973 | 17126164 | 0.91 | 16565325 | 0.97 |
| MeRIP_ko_input1 | 12499957 | 10441974 | 0.84 | 9882494 | 0.95 |
| MeRIP_ko_input2 | 6178443 | 5086508 | 0.82 | 4829848 | 0.95 |
| MeRIP_wt_IP1 | 18914882 | 17406075 | 0.92 | 16885629 | 0.97 |
| MeRIP_wt_IP2 | 14850481 | 13637258 | 0.92 | 13225871 | 0.97 |
| MeRIP_ko_IP1 | 27192835 | 23529702 | 0.87 | 22625370 | 0.96 |
| MeRIP_ko_IP2 | 13972821 | 11987963 | 0.86 | 11512812 | 0.96 |
| Ythdf1_RIP_input1 | 23419684 | 22503004 | 0.96 | 21831779 | 0.97 |
| Ythdf1_RIP_input1 | 30933222 | 29772784 | 0.96 | 28887892 | 0.97 |
| Ythdf1_RIP_IP1 | 17808787 | 11689651 | 0.66 | 11215830 | 0.96 |
| Ythdf1_RIP_IP2 | 14697186 | 9577188 | 0.65 | 9189335 | 0.96 |
| Ribo_wt_input1 | 14353853 | 14000072 | 0.98 | 9840118 | 0.70 |
| Ribo_wt_input2 | 16978427 | 16560575 | 0.98 | 11572726 | 0.70 |
| Ribo_ko_input1 | 7997730 | 7400355 | 0.93 | 4650859 | 0.63 |
| Ribo_ko_input2 | 8515757 | 7813383 | 0.92 | 4754357 | 0.61 |
| Ribo_wt_ip1 | 13096471 | 9563047 | 0.73 | 3926799 | 0.41 |
| Ribo_wt_ip2 | 9591022 | 7259191 | 0.76 | 2949562 | 0.41 |
| Ribo_ko_ip1 | 17761548 | 14796902 | 0.83 | 5634634 | 0.38 |
| Ribo_ko_ip2 | 16482288 | 13272302 | 0.81 | 5076220 | 0.38 |

GMDCs

| | | | | | |
|---|---|---|---|---|---|
| MeRIP_wt_input1 | 28551565 | 26514705 | 0.93 | 19204549 | 0.72 |
| MeRIP_wt_input2 | 28528986 | 26516721 | 0.93 | 18936050 | 0.71 |
| MeRIP_ko_input1 | 29030942 | 26613287 | 0.92 | 19307132 | 0.73 |
| MeRIP_ko_input2 | 27169760 | 24956116 | 0.92 | 18167243 | 0.73 |
| MeRIP_wt_IP1 | 28291093 | 27748060 | 0.98 | 24896670 | 0.90 |
| MeRIP_wt_IP2 | 34180741 | 33535024 | 0.98 | 30095313 | 0.90 |
| MeRIP_ko_IP1 | 22708693 | 22151135 | 0.98 | 19858221 | 0.90 |
| MeRIP_ko_IP2 | 26732331 | 26048436 | 0.97 | 23372187 | 0.90 |
| Ribo_wt_input1 | 12475501 | 9899575 | 0.79 | 4213730 | 0.43 |
| Ribo_wt_input2 | 11953956 | 9555210 | 0.80 | 4051006 | 0.42 |
| Ribo_ko_input1 | 10430201 | 8379582 | 0.80 | 3771784 | 0.45 |
| Ribo_ko_input2 | 13114316 | 10588500 | 0.81 | 4699991 | 0.44 |
| wt_monosome1 | 10968673 | 6606682 | 0.60 | 2408519 | 0.36 |
| wt_monosome2 | 10725649 | 4128146 | 0.38 | 1865605 | 0.45 |
| ko_monosome1 | 6340117 | 2504585 | 0.40 | 967011 | 0.39 |
| ko_monosome2 | 7607352 | 3063659 | 0.40 | 1496955 | 0.49 |
| wt_polysome1 | 5970349 | 2371023 | 0.40 | 865580 | 0.37 |
| wt_polysome2 | 6197195 | 2356148 | 0.38 | 1085431 | 0.46 |
| ko_polysome1 | 9760993 | 2883708 | 0.30 | 1090299 | 0.38 |
| ko_polysome2 | 7984928 | 2758005 | 0.35 | 963418 | 0.35 |
| Ythdf1_RIP_input1 | 15929824 | 15520067 | 0.97 | 13443554 | 0.87 |
| Ythdf1_RIP_input1 | 14831765 | 14546176 | 0.98 | 12629905 | 0.87 |
| Ythdf1_RIP_IP1 | 11578432 | 8250988 | 0.71 | 8028954 | 0.97 |
| Ythdf1_RIP_IP2 | 10779780 | 7371189 | 0.68 | 7178380 | 0.97 |

Sequences.

The following nucleic acids are provided by the present disclosure, as referenced herein.

(SEQ ID NO: 1)
CACCTGAGTTCAGATCATTAC
(SEQ ID NO: 2)
GCTCCAGACTGTTCATCC
(SEQ ID NO: 3)
AGATCGGAAGAGCACACGTCTGAACTCCAGTCAC
(SEQ ID NO: 4)
CTGCTTACCATACACCAT
(SEQ ID NO: 5)
TCCTTCACACTGTTAGAC
(SEQ ID NO: 6)
GGCAAGAGGTATCAAGGT
(SEQ ID NO: 7)
CAGGTAGAAGGAGAAGATGT
(SEQ ID NO: 8)
GAGTTCGCTGTGGCTAAT
(SEQ ID NO: 9)
GAGGTTCTTGCTGCTACA
(SEQ ID NO: 10)
ACCTGCCAAGTATGATGA
(SEQ ID NO: 11)
GGAGTTGCTGTTGAAGTC

It will be readily apparent to those skilled in the art that other suitable modifications

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

What is claimed is:

1. A method for enhancing cancer immunotherapy treatment comprising attenuating activity of YTH N6-Methyladenosine RNA Binding Protein 1 (YTHDF1) in a subject receiving treatment with an anticancer agent.

2. The method of claim 1, wherein YTHDF1 activity is attenuated in an antigen presenting cell (APC).

3. The method of claim 1, wherein YTHDF1 activity is attenuated in a dendritic cell (DC).

4. The method of claim 1, wherein attenuation of YTHDF1 activity reduces expression of one or more lysosomal cathepsins.

5. The method of claim 4, wherein the one or more lysosomal cathepsins comprise cathepsins B, D, and/or L.

6. The method of claim 1, wherein the anticancer agent is an immune checkpoint inhibitor.

7. The method of claim 6, wherein the immune checkpoint inhibitor is at least one of a PD-L1 antibody, a PD-1 antibody, a CTLA4 antibody, a CSG1 antibody, an IDO inhibitor, Pembrolizumab (Keytruda), Nivolumab (Opdivo), Cemiplimab (Libtayo), Atezolizumab (Tecentriq), Avelumab (Bavencio), Durvalumab (Imfinzi), and Ipilimumab (Yervoy).

8. The method of claim 1, wherein YTHDF1 activity is attenuated using at least one of antibodies and any derivatives thereof, antibody-drug conjugates, fusion proteins, small molecules, dsRNA, siRNA, anti-sense technology, aptamers, and gene editing technology (e.g., CRISPR-based methods).

9. The method of claim 1, wherein the subject has been diagnosed as having a type of cancer selected from the group consisting of melanoma, breast cancer, lung cancer, ovarian cancer, brain cancer, liver cancer, cervical cancer, colon cancer, colorectal cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, and leukemia.

10. A composition for treating cancer comprising:
an anticancer agent; and
a YTH N6-Methyladenosine RNA Binding Protein 1 (YTHDF1) inhibitor.

11. The composition of claim 10, wherein the anticancer agent is an immune checkpoint inhibitor selected from a PD-L1 antibody, a PD-1 antibody, a CTLA4 antibody, a CSG1 antibody, an IDO inhibitor, Pembrolizumab (Keytruda), Nivolumab (Opdivo), Cemiplimab (Libtayo), Atezolizumab (Tecentriq), Avelumab (Bavencio), Durvalumab (Imfinzi), and Ipilimumab (Yervoy).

12. The composition of claim 10, wherein the YTHDF1 inhibitor is selected from antibodies and any derivatives thereof, antibody-drug conjugates, fusion proteins, small molecules, dsRNA, siRNA, anti-sense technology, aptamers, and gene editing technology (e.g., CRISPR-based methods).

13. The composition of claim 10, wherein the composition reduces expression of one or more lysosomal cathepsins.

14. A method for enhancing cancer immunotherapy treatment comprising attenuating activity of one or more lysosomal cathepsins in a subject receiving treatment with an anticancer agent.

15. The method of claim 14, wherein lysosomal cathepsin activity is attenuated using at least one of antibodies and any derivatives thereof, antibody-drug conjugates, fusion proteins, small molecules, dsRNA, siRNA, anti-sense technology, aptamers, and gene editing technology (e.g., CRISPR-based methods).

16. The method of claim 14, wherein lysosomal cathepsin activity is attenuated using one or more of E64, CA-074, and CASIII.

17. The method of claim 14, wherein the one or more lysosomal cathepsins comprise cathepsins B, D, and/or L.

18. The method of claim 14, wherein the subject has been diagnosed as having a type of cancer selected from the group consisting of melanoma, breast cancer, lung cancer, ovarian cancer, brain cancer, liver cancer, cervical cancer, colon cancer, colorectal cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, and leukemia.


SEQUENCE LISTING (11 sequences)

SEQ ID NO: 1 - DNA, 21 bp, Artificial sequence
  Synthetic
cacctgagttcagatcattac

SEQ ID NO: 2 - DNA, 18 bp, Artificial sequence
  Synthetic
gctccagactgttcatcc

SEQ ID NO: 3 - DNA, 34 bp, Artificial sequence
  Synthetic
agatcggaagagcacacgtctgaactccagtcac

SEQ ID NO: 4 - DNA, 18 bp, Artificial sequence
  Synthetic
ctgcttaccatacaccat

SEQ ID NO: 5 - DNA, 18 bp, Artificial sequence
  Synthetic
tccttcacactgttagac

SEQ ID NO: 6 - DNA, 18 bp, Artificial sequence
  Synthetic
ggcaagaggtatcaaggt

SEQ ID NO: 7 - DNA, 20 bp, Artificial sequence
  Synthetic
caggtagaaggagaagatgt

SEQ ID NO: 8 - DNA, 18 bp, Artificial sequence
  Synthetic
gagttcgctgtggctaat

SEQ ID NO: 9 - DNA, 18 bp, Artificial sequence
  Synthetic
gaggttcttgctgctaca

SEQ ID NO: 10 - DNA, 18 bp, Artificial sequence
  Synthetic
acctgccaagtatgatga

SEQ ID NO: 11 - DNA, 18 bp, Artificial sequence
  Synthetic
ggagttgctgttgaagtc